(19) United States
(12) Patent Application Publication
(10) Pub. No.: US 2025/0271446 A1
(43) Pub. Date: Aug. 28, 2025

(54) Combination Of Proteotypic Peptides For The Detection Of Proteins In The Pd-L1 Axis

(71) Applicants: The Royal Institution for the Advancement of Learming/McGill University, Montreal (CA); Sir Mortimer B. Davis Jewish General Hospital, Montreal (CA)

(72) Inventors: Christoph Hermann Borchers, Montreal (CA); Alan Spatz, Westmount (CA); Réne Peiman Zahedi, Laval, CA (US); Vincent Roy Richard, Montreal, CA (US); Nancy Fournier, Montreal (CA); Mounib ELCHEBLY, Montreal (CA); Vincent Lacasse, Saint-Amable (CA)

(21) Appl. No.: 18/560,387

(22) PCT Filed: May 18, 2022
(86) PCT No.: PCT/CA2022/050783

§ 371 Date: Nov. 12, 2023

Related U.S. Application Data
(60) Provisional application No. 63/190,861, filed on May 20, 2021.

(51) Int. Cl. G01N 33/68; C07K 16/28; G01N 33/574

(52) U.S. Cl. CPC G01N 33/6848; C07K 16/2818; C07K 16/2827; G01N 33/57423; G01N 2333/70521; G01N 2333/70532; G01N 2333/70596; G01N 2333/912

(57) ABSTRACT

The present disclosure concerns a combination comprising a plurality of proteotypic peptides for measuring, using mass spectrometry, the expression of at least two proteins in the PD-L1 axis. The combination comprises a synthetic standard lymphocyte specific kinase (LCK) peptide, a synthetic standard NT5E (CD73) peptide, a synthetic standard programmed cell death protein 1 (PD-1) peptide, a synthetic standard programmed death-ligand 1 (PD-1) peptide, a synthetic standard programmed death-ligand 2 (PD-L2) peptide and/or a synthetic standard zeta-chain-associated protein kinase 70 (ZAP70) peptide. The combination of synthetic standard peptides can be used in combination with affinity agents, such as antibodies, specific for synthetic standard peptides. The combination of the present disclosure can be used to tailor an appropriate therapeutic regimen or monitor the efficiency of a therapeutic regimen comprising checkpoint inhibitor modulating agents.

### CROSS-REFERENCE TO RELATED APPLICATIONS

This application is a national phase entry of PCT/CA2022/050783 filed on May 18, 2022, which claims priority from U.S. Ser. No. 63/190,861 filed on May 20, 2021, each incorporated herein by reference in their entirety.

### INCORPORATION BY REFERENCE OF SEQUENCE LISTING

The application herein contains a sequence listing in electronic, ASCII format. The content of this sequence listing is incorporated herein in its entirety. The ASCII copy, created on May 14, 2022, is named Sequence listing Proteotypic Peptides_ST25 and is 6000 bytes in size.

### TECHNOLOGICAL FIELD

The present disclosure relates to proteotypic peptides that can be used for quantifying, using mass spectrometry, one or more proteins in a programmed death-ligand 1 (PD-L1) axis.

### BACKGROUND

Proteins of the programmed death-ligand 1 (PD-L1) axis are associated with immune function and they play a central role in various autoimmune diseases and cancers. A variety of therapeutics, including antibodies that target checkpoint inhibitors (CPIs) and restore anti-tumour immune response, have revolutionized lung cancer treatment but up to 50% of patients that show positive PD-L1 expression by immunohistochemistry (IHC) do not respond to anti-PD-L1 treatment, and some patients with low/undetectable PD-L1 levels have significantly improved survival with checkpoint inhibitors. Moreover, PD-L1 immunohistochemistry (IHC), which is the clinically used method to assess PD-L1 expression in tumor tissue, is non-standardized, heterogeneous, and can be affected by tissue fixation time and post-translational modifications (e.g., glycosylation).

Additionally, multiple studies indicate that the expression of PD-L1 alone does not reliably reflect the tumour immune microenvironment, thus necessitating the measurement of other members of the PD-1 axis or signalling pathway.

It would be highly desirable to provide reagents and methods for quantifying one or more proteins in the PD-L1 axis. In some embodiments, it would be highly desirable to provide reagents and methods for quantifying concurrently (in a multiplex format) more than one protein in the PD-L1 axis. In some further embodiments, it would be highly desirable to provide reagents and methods for use with samples in various formats (fluids, tissues, cells, etc.), which includes both those that may have been pre-treated (fixed, for example) or untreated.

### BRIEF SUMMARY

According to a first aspect, the present disclosure provides a combination of proteotypic peptides for quantitative mass spectrometry for at least one protein in a programmed death-ligand 1 (PD-L1) axis. The at least one protein in the PD-L1 axis is lymphocyte specific kinase (LCK), NT5E (CD73), programmed cell death protein 1 (PD-1), programmed death-ligand 1 (PD-L1), programmed death-ligand 2 (PD-L2) or zeta-chain-associated protein kinase 70 (ZAP70). The combination comprises at least two of the following synthetic standard peptides: (i) a synthetic standard lymphocyte specific kinase (LCK) peptide: ESESTAGSFSLSVR (SEQ ID NO: 1), ITFPGLHELVR (SEQ ID NO: 2) and/or QLLAPGNTHGSFLIR (SEQ ID NO: 3); (ii) a synthetic standard NT5E (CD73) peptide: GPLASQISGLYLPYK (SEQ ID NO: 4) and/or HDSGDQDINVVSTYISK (SEQ ID NO: 5); (iii) a synthetic standard programmed cell death protein 1 (PD-1) peptide: EDPSAVPVFSVDYGELDFQWR (SEQ ID NO: 6); LAAFPEDR (SEQ ID NO: 7); SQPGQDCR (SEQ ID NO: 8); VTQLPNGR (SEQ ID NO: 9); DDSGTYLCGAISLAPK (SEQ ID NO: 10) and/or NDSGTYLCGAISLAPK (SEQ ID NO: 11); (iv) a synthetic standard programmed death-ligand 1 (PD-L1) peptide: ILVVDPVTSEHELTCQAEGYPK (SEQ ID NO: 12); LFDVTSTLR (SEQ ID NO: 13); LFNVTSTLR (SEQ ID NO: 14); LQDAGVYR (SEQ ID NO: 15); NIIQFVHGEEDLK (SEQ ID NO: 16); INTTTNEIFYCTFR (SEQ ID NO: 17) and/or IDTTTNEIFYCTFR (SEQ ID NO: 18); (v) a synthetic standard programmed death-ligand 2 (PD-L2) peptide: ASFHIPQVQVR (SEQ ID NO: 19); ATLLEEQLPLGK (SEQ ID NO: 20), TPEGLYQVTSVLR (SEQ ID NO: 21); NFSCVFWNTHVR (SEQ ID NO: 22) and/or DFSCVFWNTHVR (SEQ ID NO: 23); or (vi) a synthetic standard zeta-chain-associated protein kinase 70 (ZAP70) peptide: LEGEALEQAIISQAPQVEK (SEQ ID NO: 24); LIATTAHER (SEQ ID NO: 25) and/or SLGGYVLSLVHDVR (SEQ ID NO: 26). In an embodiment, the combination comprises synthetic standard peptides specific for at least two proteins in the PD-L1 axis. In some embodiments, each of the synthetic standard peptides comprise an epitope that is present on the corresponding protein in the PD-L1 axis. In yet an additional embodiment, the combination comprises the synthetic standard PD-L1 peptide. In still another embodiment, the combination comprises unlabeled synthetic standard peptides, isotope-labeled synthetic standard peptides, or a combination of both. In still yet another embodiment, the combination further comprises at least one endogenous peptide obtained from the proteolytic digestion of LCK, NT5E, PD-1, PD-L1, PD-L2 or ZAP70. In some embodiments, the combination comprises a deglycosylated proteotypic standard synthetic peptide and/or a deglycosylated endogenous peptide. In another embodiment, the quantitative mass spectrometry is a multiple reaction monitoring mass spectrometry, a parallel reaction monitoring mass spectrometry, a matrix-assisted laser desorption/ionization (MALDI) mass spectrometry, or a data independent acquisition mass spectrometry. In still another embodiment, the multiple reaction monitoring mass spectrometry is an immuno-multiple reaction monitoring mass spectrometry, an immuno-parallel reaction monitoring, an immuno-MALDI, or an immuno-data independent acquisition mass spectrometry.

According to a second aspect, the present disclosure provides an antibody combination for use as affinity agents prior to a quantitative mass spectrometry. The antibody combination comprises at least two antibodies and each antibody is able to bind to a different synthetic standard peptide described herein. In an embodiment, at least one antibody of the combination has been obtained by eliciting antibody production in a subject having been administered an immunogen comprising the amino acid sequence of a synthetic standard peptide described herein. In yet another embodiment, the antibody combination comprises at least one monoclonal antibody, a polyclonal antibody or a single domain antibody. In some embodiments, the quantitative mass spectrometry is a multiple reaction monitoring mass spectrometry, a parallel reaction monitoring mass spectrometry, a matrix-assisted laser desorption/ionization (MALDI) mass spectrometry, or a data-independent acquisition mass spectrometry. In additional embodiments, the multiple reaction monitoring mass spectrometry is an immuno-multiple reaction monitoring mass spectrometry, an immuno-parallel reaction monitoring, an immuno-MALDI, or an immuno-data independent acquisition mass spectrometry.

According to a third aspect, the present disclosure provides a kit comprising the combination described herein and the antibody combination described herein.

According to a fourth aspect, the present disclosure provides a method of performing a quantitative mass spectrometry to determine the abundance of at least two proteins in a programmed death-ligand 1 (PD-L1) axis in a biological sample from a subject. The method comprises: a) obtaining a biological sample suspected of comprising endogenous peptides obtained from the proteolytic digestion of the at least two proteins in the PD-L1 axis; b) combining the sample with a combination of at least two synthetic standard peptides, each synthetic standard peptide being specific for a different protein in the PD-L1 axis to obtain a supplemented mixture; c) enriching the supplemented mixture with a combination of at least two affinity agents, wherein the combination comprises at least one affinity agent being specific for each synthetic standard peptide to obtain an enriched mixture; and d) submitting the enriched mixture to the quantitative mass spectrometry to determine the abundance of the at least two proteins in the PD-L1 axis. The proteins in the PD-L1 axis comprise LCK, NT5E, PD-1, PD-L1, PD-L2 and ZAP70. In an embodiment, the at least two synthetic standard peptides are described herein. In another embodiment, the combination of the at least two affinity agents comprises at least two antibodies. In yet another embodiment, the combination of the at least two antibodies is described herein. In some embodiments, the method further comprises, prior to step a), obtaining the biological sample from the subject. In yet another embodiment, the method further comprises, prior to step a), enzymatically treating the biological sample from the subject to obtain the endogenous peptides. In yet still another embodiment, the method further comprises, before step c), deglycosylating the synthetic standard peptides and/or the endogenous peptides. In embodiments, the biological sample comprises a fluid, a cell or a tissue. In further embodiments, the biological sample comprises a living cell, a frozen cell or a fixed cell. In additional embodiments, the biological sample is suspected of comprising a cancerous cell. In some embodiments, the quantitative mass spectrometry is a multiple reaction monitoring mass spectrometry, a parallel reaction monitoring mass spectrometry, a matrix-assisted laser desorption/ionization (MALDI) mass spectrometry or a data-independent acquisition mass spectrometry. In yet further embodiments, the multiple reaction monitoring mass spectrometry is an immuno-multiple reaction monitoring mass spectrometry, an immuno-parallel reaction monitoring, an immuno-MALDI, or an immuno-data independent acquisition mass spectrometry.

According to a fifth aspect, the present disclosure provides a method of identifying a subject that will respond to an immune checkpoint inhibitor or an immune checkpoint inhibiting agent. The method comprises determining the abundance of at least two proteins in a programmed death-ligand 1 (PD-L1) axis in a biological sample of the subject and administering the immune checkpoint inhibitor or the immune checkpoint activating agent to the subject if it has been determined that the biological sample comprises a modulation in the expression of at least two proteins in the PD-L1 axis. The proteins in the PD-L1 axis comprise LCK, NT5E, PD-1, PD-L1, PD-L2 and ZAP70. In an embodiment, the condition is a cancer. In another embodiment, the condition is a solid cancer. In still another embodiment, the solid cancer is lung cancer. In some embodiments, the method comprises using the combination described herein, the antibody combination described herein or the kit described herein or performing the method described herein to determine the abundance of the at least two proteins in a programmed death-ligand 1 (PD-L1) axis in the biological sample of the subject. In an embodiment, the subject did not previously receive administering the immune checkpoint inhibitor or the immune check-point inhibitor activating agent. In another embodiment, the subject did previously receive administering the immune checkpoint inhibitor or the immune check-point inhibitor activating agent. In some embodiments, the immune checkpoint inhibitor activating agent comprises an antagonistic antibody. In some further embodiments, the immune checkpoint inhibitor activating agent comprises an anti-programmed cell death 1 (PD-1) antibody, an anti-programmed cell death 1 ligand (PD-L1) antibody, and/or an anti-programmed cell death 2 ligand (PD-L2) antibody.

According to a sixth aspect, the present disclosure provides a method of treating a subject having or suspected to have a condition associated with a modulation in the expression of a protein in a programmed death-ligand 1 (PD-L1) axis. The method comprising determining the abundance of at least two proteins in a programmed death-ligand 1 (PD-L1) axis in a biological sample of the subject and administering an immune checkpoint inhibitor or an immune checkpoint inhibitor activating agent to the subject if it has been determined that the biological sample comprises a modulation in the expression of at least two proteins in the PD-L1 axis. The proteins in the PD-L1 axis comprises LCK, NT5E, PD-1, PD-L1, PD-L2 and ZAP70. In an embodiment, the condition is a cancer. In another embodiment, the condition is a solid cancer. In still another embodiment, the solid cancer is lung cancer. In some embodiments, the method comprises using the combination described herein, the antibody combination described herein or the kit described herein or performing the method described herein to determine the abundance of the at least two proteins in a programmed death-ligand 1 (PD-L1) axis in the biological sample of the subject. In an embodiment, the subject did not previously receive administering the immune checkpoint inhibitor or the immune check-point inhibitor activating agent. In another embodiment, the subject did previously receive administering the immune checkpoint inhibitor or the immune check-point inhibitor activating agent. In some embodiments, the immune checkpoint inhibitor activating agent comprises an antagonistic antibody. In some further embodiments, the immune checkpoint inhibitor activating agent comprises an anti-programmed cell death 1 (PD-1) antibody, an anti-programmed cell death 1 ligand (PD-L1) antibody, and/or an anti-programmed cell death 2 ligand (PD-L2) antibody.

### BRIEF DESCRIPTION OF THE DRAWINGS

Having thus generally described the nature of the invention, reference will now be made to the accompanying drawings, showing by way of illustration, a preferred embodiment thereof, and in which:

FIGS. 1A to 1C. Quantitation in the PD-L1 axis in ‘immune-cold’ (low response to CPI) colorectal cancer (CRC) tumors using multiplexed immuno-multiple reaction monitoring (MRM) mass spectrometry.

FIG. 1A Proteins were extracted from formalin-fixed paraffin-embedded (FFPE) cores. After proteolytic digestion, stable-isotope labeled standard (SIS) peptides were spiked in known amounts.

FIG. 1B PD-L1, PD-L2, PD-1, LCK, ZAP70, and NT5E (CD 73) anti-peptide antibody-bead conjugates were mixed and incubated with the tumor tissue digest for enrichment of the target peptides. Upon washing and removal of the background, the bound peptides were eluted and the obtained eluates were subsequently analyzed by multiple reaction monitoring (MRM) mass spectrometry. The ratios of endogenous and SIS peptide MRM signal areas were used to determine the absolute concentrations of the target proteins.

FIG. 1C The concentration of PD-L1 axis protein concentrations for seven different PDX-derived FFPE blocks (P1-P8) was determined. Per block, three cores per punched to address tumor heterogeneity. Per punch, a total of 70 μg of total protein was used. Average concentrations and standard deviations are given. Each PDX was screened for driver mutations by Nanostring sequencing (red cells). nd=not detected; *=only quantified in one core, no standard deviation; **=below limit of quantification.

FIG. 2 provides a description of the biological sequences of the present disclosure.

FIG. 3A shows the quantification by immuno-multiple reaction monitoring (iMRM) of each peptide in the PD-1/PD-L1 axis in formalin-fixed paraffin-embedded (FFPE) non-small cell lung cancer (NSCLC) samples (n=18).

FIG. 3B shows the quantification by iMRM of each peptide in the PD-1/PD-L1 axis in samples frozen while fresh (fresh frozen) NSCLC samples (n=32).

FIG. 3C shows weight-normalized iMRM quantification of each peptide in the PD-1/PD-L1 axis in FFPE NSCLC samples (n=18).

FIG. 3D shows weight-normalized iMRM quantification of each peptide in the PD-1/PD-L1 axis in fresh frozen NSCLC samples (n=32).

FIG. 3E shows the correlation between two PD-L1 peptides quantified by iMRM in FFPE NSCLC samples.

FIG. 3F shows the correlation between two PD-L1 peptides quantified by iMRM in fresh frozen NSCLC samples.

FIG. 4A is an equation for the calculation for the immunoscore, where I is the immunoscore, and α (ATLL), β (LFDV), γ (LQDA), δ (ITFP) and ε (LIAT) equal 1 if the peptide concentration is above the cutoff or −1 if it is below the cut-off.

FIG. 4B is a Kaplan-Meier curve for patients with an immune-high (immunoscore ≥0) vs immune-low profile (immunoscore <0).

### DETAILED DESCRIPTION

The present disclosure provides a multiplexed assay for determining the level of expression of at least two different proteins in the PD-L1 axis. The assay is based on the use of a combination of synthetic standard peptides for quantitative mass spectrometry. The assay can also include the use of a combination of affinity agents (such as antibodies, beads/particles, and the like). Without wishing to be bound to theory, the synthetic versions of the proteotypic peptides described herein can be used as internal standards to facilitate the standardization of the assay to provide more reliable results (e.g., level of expression, fully quantifiable results) and the comparison of the results obtained between laboratory sites. In some embodiments, the assay is precise within its linear range and can even provide lower-limits-of-quantification (sub-fmol range) and/or lower-limits-of-detection that are below those of existing assays. In some further embodiments, the assay can be designed to detect and quantify different isoforms of the proteins in the PD-L1 axis, as well as the extent of their site-specific glycosylation.

### Proteotypic Peptides

The assay described herein is based on the use of a combination of synthetic standard peptides for quantifying the amount/expression of a plurality of proteins in the PD-L1 axis of a sample obtained or derived from a human subject. Each synthetic standard peptide is proteotypic for one protein in the PD-L1 axis. In the context of the present disclosure, a proteotypic peptide refers to a peptide having a unique amino acid sequence within the genome, corresponding to endogenous peptides capable of being obtained efficiently using enzymatic digestion (e.g., proteolysis) of a protein in the PD-L1 axis and/or can be detected by mass spectrometry with high sensitivity and specificity. In some embodiments, the synthetic standard peptide is not substantially modified during the sample preparation (e.g., it lacks or has a limited amount of methionine residues which can oxidized and/or it lacks or has a limited amount of asparagine and/or glycine residues which can be deamidated). In some additional embodiments, the synthetic standard peptides of the present disclosure can be deaminated (Asn→Asp) prior to or during sample preparation.

As indicated herein, the synthetic standard peptides described herein can be used in combination with one or more affinity agents (e.g., antibodies). The affinity agents are selected or designed based on their ability to recognize and bind to the synthetic standard peptide. As such, in embodiments in which the affinity agent is an antibody, the synthetic standard peptide comprises an epitope (recognized by the antibody) which is also present on the corresponding endogenous peptide (e.g., an enzymatic (proteolytic) fragment of a protein in the PD-L1 axis). In some embodiments, the synthetic standard peptide of the present disclosure (either alone or in the form of an immunogen) has the ability to generate an antibody in a subject (which can be, for example, an animal, such as a rodent, including but not limited to a rabbit, a mouse or a rat) and such an antibody is specific for the corresponding endogenous peptide.

The combination of synthetic standard peptides in the present disclosure include at least two different peptides, each peptide being proteotypic for a distinct protein in the PD-L1 axis. In the context of the present disclosure, the PD-L1 axis includes the following proteins: a lymphocyte specific kinase (LCK), NT5E (CD73), a programmed cell death protein 1 (PD-1), a programmed death-ligand 1 (PD-L1), a programmed death-ligand 2 (PD-L2) and a zeta-chain-associated protein kinase 70 (ZAP70). In some embodiments, the combination of synthetic standard peptides in the present disclosure can include at least three peptides, each peptide being proteotypic for a distinct protein in the PD-L1 axis. In some embodiments, the combination of the synthetic standard peptides of the present disclosure can include at least four peptides, each peptide being proteotypic for a distinct protein in the PD-L1 axis. In some embodiments, the combination of the synthetic standard peptides of the present disclosure can include at least five peptides, each peptide being proteotypic for a distinct protein in the PD-L1 axis. In some embodiments, the combination of the synthetic standard peptides of the present disclosure can include at least six peptides, each peptide being proteotypic for a distinct protein in the PD-L1 axis. In some embodiments, the combination of synthetic standard peptides can include more than one peptide specific for the same protein in the PD-L1 axis. Table 1 below provides some embodiments of the classes of synthetic standard peptides which can be present in the combination of the present disclosure.

TABLE 1
Classes of synthetic standard peptides
that can be present in the combination.

| | Syn- | Syn- | Syn- | Syn- | Syn- | Syn- |
|---|---|---|---|---|---|---|
| | thetic | thetic | thetic | thetic | thetic | thetic |
| | standard | standard | standard | standard | standard | standard |
| | LCK | NT5E | PD-1 | PD-L1 | PD-L2 | ZAP70 |
| # | peptide | peptide | peptide | peptide | peptide | peptide |
| 1 | + | + | | | | |
| 2 | | + | + | | | |
| 3 | | | + | + | | |
| 4 | | | | + | + | |
| 5 | | | | | + | + |
| 6 | + | | + | | | |
| 7 | | + | | + | | |
| 8 | | | + | | + | |
| 9 | | | | + | | + |
| 10 | + | | | + | | |
| 11 | | + | | | + | |
| 12 | | | + | | | + |
| 13 | + | | | | + | |
| 14 | | + | | | | + |
| 15 | + | | | | | + |
| 16 | + | + | + | | | |
| 17 | | + | + | + | | |
| 18 | | | + | + | + | |
| 19 | | | | + | + | + |
| 20 | + | + | | + | | |
| 21 | | + | + | | + | |
| 22 | | | + | + | | + |
| 23 | + | + | | | + | |
| 24 | | + | + | | | + |
| 25 | + | + | | | | + |
| 26 | + | | + | + | | |
| 27 | | + | | + | + | |
| 28 | | | + | | + | + |
| 29 | + | | + | | + | |
| 30 | | + | | + | | + |
| 31 | + | | + | | | + |
| 32 | + | | | + | + | |
| 33 | | + | | | + | + |
| 34 | + | | | + | | + |
| 35 | + | | | | + | + |
| 36 | + | + | + | + | | |
| 37 | | + | + | + | + | |
| 38 | | | + | + | + | + |
| 39 | + | + | + | | + | |
| 40 | | + | + | + | | + |
| 41 | + | + | + | | | + |
| 42 | + | + | | + | + | |
| 43 | | + | + | | + | + |
| 44 | + | + | | + | | + |
| 45 | + | + | | | + | + |
| 46 | + | | + | + | + | |
| 47 | | + | | + | + | + |
| 48 | + | | + | + | | + |
| 49 | + | | + | | + | + |
| 50 | + | | | + | + | + |
| 51 | + | + | + | + | + | |
| 52 | + | | + | + | + | + |
| 53 | + | + | | + | + | + |
| 54 | + | + | + | | + | + |
| 55 | + | + | + | + | | + |
| 56 | | + | + | + | + | + |
| 57 | + | + | + | + | + | + |

+ indicates that the class of synthetic standard peptide is present whereas the absence of “+” indicates that the class of synthetic standard peptide is absent.

In one embodiment, the combination comprises at least one synthetic standard LCK peptide. In some additional embodiments, the combination comprises at least two synthetic standard LCK peptides. In some further embodiments, the combination comprises at least three synthetic standard LCK peptides. Table 2 below provides some embodiments of the specific LCK synthetic standard peptides which can be present in the combination of the present disclosure.

TABLE 2
LCK synthetic standard peptides that
can be present in the combination.

| # | SEQ ID NO: 1 | SEQ ID NO: 2 | SEQ ID NO: 3 |
|---|---|---|---|
| 58 | + | | |
| 59 | | + | |
| 60 | | | + |
| 61 | + | + | |
| 62 | | + | + |
| 63 | + | | + |
| 64 | + | + | + |

+ indicates that the synthetic standard peptide is present whereas the absence of “+” indicates that the proteotypic peptide is absent.

In one embodiment, the combination does not include the synthetic standard lymphocyte specific kinase (LCK) peptide having the amino acid sequence of ESESTAGSFSLSVR (SEQ ID NO: 1). In such an embodiment, the synthetic standard lymphocyte specific kinase (LCK) peptide present in the combination could have the amino acid sequence of ITFPGLHELVR (SEQ ID NO: 2), QLLAPGNTHGSFLIR (SEQ ID NO: 3), alone or in combination with one another.

In another embodiment, the combination comprises at least one synthetic standard NT5E peptide. In some additional embodiments, the combination comprises at least two synthetic standard NT5E peptide. Table 3 below provides some embodiments of the specific NT5E synthetic standard peptides which can be present in the combination of the present disclosure.

TABLE 3
NT5E synthetic standard peptides that
can be present in the combination.

| # | SEQ ID NO: 4 | SEQ ID NO: 5 |
|---|---|---|
| 65 | + | |
| 66 | | + |
| 67 | + | + |

+ indicates that the synthetic standard peptide is present whereas the absence of “+” indicates that the synthetic standard peptide is absent.

In one embodiment, the combination does not include the synthetic standard NT5E peptide having the amino acid sequence GPLASQISGLYLPYK (SEQ ID NO: 4). In such an embodiment, the synthetic standard NT5E peptide present in the combination could have the amino acid sequence of HDSGDQDINVVSTYISK (SEQ ID NO: 5). In another embodiment, the combination does not include the synthetic standard NT5E peptide having the amino acid sequence HDSGDQDINVVSTYISK (SEQ ID NO: 5). In such an embodiment, the synthetic standard NT5E peptide present in the combination could have the amino acid sequence of GPLASQISGLYLPYK (SEQ ID NO: 4).

In still another embodiment, the combination comprises at least one synthetic standard PD-1 peptide. In some additional embodiments, the combination comprises at least two synthetic standard PD-1 peptides. In some further embodiments, the combination comprises at least three synthetic standard PD-1 peptides. In some additional embodiments, the combination comprises at least four synthetic standard PD-1 peptides. In some further embodiments, the combination comprises at least five synthetic standard PD-1 peptides. In some further embodiments, the combination comprises at least six synthetic standard PD-1 peptides. Table 4 below provides some embodiments of the specific PD-1 synthetic standard peptides which can be present in the combination of the present disclosure.

TABLE 4
PD-1 synthetic standard peptides that
could be present in the combination.

| | SEQ ID | SEQ ID | SEQ ID | SEQ ID | SEQ D | SEQ ID |
|---|---|---|---|---|---|---|
| # | NO: 6 | NO: 7 | NO: 8 | NO: 9 | NO: 10 | NO: 11 |
| 68 | + | + | | | | |
| 69 | | + | + | | | |
| 70 | | | + | + | | |
| 71 | | | | + | + | |
| 72 | | | | | + | + |
| 73 | + | | + | | | |
| 74 | | + | | + | | |
| 75 | | | + | | + | |
| 76 | | | | + | | + |
| 77 | + | | | + | | |
| 78 | | + | | | + | |
| 79 | | | + | | | + |
| 80 | + | | | | + | |
| 81 | | + | | | | + |
| 82 | + | | | | | + |
| 83 | + | + | + | | | |
| 84 | | + | + | + | | |
| 85 | | | + | + | + | |
| 86 | | | | + | + | + |
| 87 | + | + | | + | | |
| 88 | | + | + | | + | |
| 89 | | | + | + | | + |
| 90 | + | + | | | + | |
| 91 | | + | + | | | + |
| 92 | + | + | | | | + |
| 93 | + | | + | + | | |
| 94 | | + | | + | + | |
| 95 | | | + | | + | + |
| 96 | + | | + | | + | |
| 97 | | + | | + | | + |
| 98 | + | | + | | | + |
| 99 | + | | | + | + | |
| 100 | | + | | | + | + |
| 101 | + | | | + | | + |
| 102 | + | | | | + | + |
| 103 | + | + | + | + | | |
| 104 | | + | + | + | + | |
| 105 | | | + | + | + | + |
| 106 | + | + | + | | + | |
| 107 | | + | + | + | | + |
| 108 | + | + | + | | | + |
| 109 | + | + | | + | + | |
| 110 | | + | + | | + | + |
| 111 | + | + | | + | | + |
| 112 | + | + | | | + | + |
| 113 | + | | + | + | + | |
| 114 | | + | | + | + | + |
| 115 | + | | + | + | | + |
| 116 | + | | + | | + | + |
| 117 | + | | | + | + | + |
| 118 | + | + | + | + | + | |
| 119 | + | | + | + | + | + |
| 120 | + | + | | + | + | + |
| 121 | + | + | + | | + | + |
| 122 | + | + | + | + | | + |
| 123 | | + | + | + | + | + |
| 124 | + | + | + | + | + | + |

+ indicates that the synthetic standard peptide is present whereas the absence of “+” indicates that the synthetic standard peptide is absent.

In another embodiment, the combination does not include the synthetic standard PD-1 peptide having the amino acid sequence of LAAFPEDR (SEQ ID NO: 7). In this embodiment, the synthetic standard PD-1 peptide present in the combination can have the amino acid sequence of EDPSAVPVFSVDYGELDFQWR (SEQ ID NO: 6), SQPGQDCR (SEQ ID NO: 8), VTQLPNGR (SEQ ID NO: 9), DDSGTYLCGAISLAPK (SEQ ID NO: 10) or NDSGTYLCGAISLAPK (SEQ ID NO: 11), alone or in any combination with each other.

In another embodiment, the combination does not include the synthetic standard PD-1 peptide having the amino acid sequence of EDPSAVPVFSVDYGELDFQWR (SEQ ID NO: 6). In this embodiment, the synthetic standard PD-1 peptide present in the combination can have the amino acid sequence of LAAFPEDR (SEQ ID NO: 7), SQPGQDCR (SEQ ID NO: 8), VTQLPNGR (SEQ ID NO: 9), DDSGTYLCGAISLAPK (SEQ ID NO: 10) or NDSGTYLCGAISLAPK (SEQ ID NO: 11), alone or in any combination with each other.

In yet another embodiment, the combination comprises at least one synthetic standard PD-L1 peptide. In some additional embodiments, the combination comprises at least two synthetic standard PD-L1 peptides. In some further embodiments, the combination comprises at least three synthetic standard PD-L1 peptides. In some additional embodiments, the combination comprises at least four synthetic standard PD-L1 peptides. In some further embodiments, the combination comprises at least five synthetic standard PD-L1 peptides. In some further embodiments, the combination comprises at least six synthetic standard PD-L1 peptides. In some further embodiments, the combination comprises at least seven synthetic standard PD-L1 peptides. Table 5 below provides some embodiments of the specific PD-L1 synthetic standard peptides which can be present in the combination of the present disclosure.

TABLE 5
PD-L1 synthetic standard peptides that
could be present in the combination.

| | SEQ ID | SEQ ID | SEQ ID | SEQ ID | SEQ D | SEQ ID | SEQ ID |
|---|---|---|---|---|---|---|---|
| # | NO: 12 | NO: 13 | NO: 14 | NO: 15 | NO: 16 | NO: 17 | NO: 18 |
| 125 | + | | | | | | |
| 126 | | + | | | | | |
| 127 | | | + | | | | |
| 128 | | | | + | | | |
| 129 | | | | | + | | |
| 130 | | | | | | + | |
| 131 | | | | | | | + |
| 132 | + | + | | | | | |
| 133 | | + | + | | | | |
| 134 | | | + | + | | | |
| 135 | | | | + | + | | |
| 136 | | | | | + | + | |
| 137 | | | | | | + | + |
| 138 | + | | + | | | | |
| 139 | | + | | + | | | |
| 140 | | | + | | + | | |
| 141 | | | | + | | + | |
| 142 | | | | | + | | + |
| 143 | + | | | + | | | |
| 144 | | + | | | + | | |
| 145 | | | + | | | + | |
| 146 | | | | + | | | + |
| 147 | + | | | | + | | |
| 148 | | + | | | | + | |
| 149 | | | + | | | | + |
| 150 | + | | | | | + | |
| 151 | | + | | | | | + |
| 152 | + | | | | | | + |
| 153 | + | + | + | | | | |
| 154 | | + | + | + | | | |
| 155 | | | + | + | + | | |
| 156 | | | | + | + | + | |
| 157 | | | | | + | + | + |
| 158 | + | + | | + | | | |
| 159 | | + | + | | + | | |
| 160 | | | + | + | | + | |
| 161 | | | | + | + | | + |
| 162 | + | + | | | + | | |
| 163 | | + | + | | | + | |
| 164 | | | + | + | | | + |
| 165 | + | + | | | | + | |
| 166 | | + | + | | | | + |
| 167 | + | + | | | | | + |
| 168 | + | | + | + | | | |
| 169 | | + | | + | + | | |
| 170 | | | + | | + | + | |
| 171 | | | | + | | + | + |
| 172 | + | | + | | + | | |
| 173 | | + | | + | | + | |
| 174 | | | + | | + | | + |
| 175 | + | | + | | | + | |
| 176 | | + | | + | | | + |
| 177 | + | | + | | | | + |
| 178 | + | | | + | + | | |
| 179 | | + | | | + | + | |
| 180 | | | + | | | + | + |
| 181 | + | | | + | | + | |
| 182 | | + | | | + | | + |
| 183 | + | | | + | | | + |
| 184 | + | | | | + | + | |
| 185 | | + | | | | + | + |
| 186 | + | | | | + | | + |
| 187 | + | | | | | + | + |
| 188 | + | + | + | + | | | |
| 189 | | + | + | + | + | | |
| 190 | | | + | + | + | + | |
| 191 | | | | + | + | + | + |
| 192 | + | + | + | | + | | |
| 193 | | + | + | + | | + | |
| 194 | | | + | + | + | | + |
| 195 | + | + | + | | | + | |
| 196 | | + | + | + | | | + |
| 197 | + | + | + | | | | + |
| 198 | + | + | | + | + | | |
| 199 | | + | + | | + | + | |
| 200 | | | + | + | | + | + |
| 201 | + | + | | + | | + | |
| 202 | | + | + | | + | | + |
| 203 | + | + | | + | | | + |
| 204 | + | + | | | + | + | |
| 205 | | + | + | | | + | + |
| 206 | + | + | | | + | | + |
| 207 | + | + | | | | + | + |
| 208 | + | | + | + | + | | |
| 209 | | + | | + | + | + | |
| 210 | | | + | | + | + | + |
| 211 | + | | + | + | | + | |
| 212 | | + | | + | + | | + |
| 213 | + | | + | + | | | + |
| 214 | + | | + | | + | | + |
| 215 | + | | + | | | + | + |
| 216 | + | | | + | + | + | |
| 217 | | + | | | + | + | + |
| 218 | + | | | + | + | | + |
| 219 | + | | | + | | + | + |
| 220 | + | | | | + | + | + |
| 221 | | | + | + | + | + | + |
| 222 | + | | | + | + | + | + |
| 223 | + | + | | | + | + | + |
| 224 | + | + | + | | | + | + |
| 225 | + | + | + | + | | | + |
| 226 | + | + | + | + | + | | |
| 227 | | + | | + | + | + | + |
| 228 | + | | + | | + | + | + |
| 229 | + | + | | + | | + | + |
| 230 | + | + | + | | | | + |
| 231 | + | + | + | + | | + | |
| 232 | | + | + | | + | + | + |
| 233 | + | | + | + | | + | + |
| 234 | + | + | | + | + | | + |
| 235 | + | + | + | | + | + | |
| 236 | | + | + | + | | + | + |
| 237 | + | | + | + | + | | + |
| 238 | + | + | | + | + | + | |
| 239 | | + | + | + | + | | + |
| 240 | + | | + | + | + | + | |
| 241 | | + | + | + | + | + | |
| 242 | + | + | + | + | + | + | |
| 243 | | + | + | + | + | + | + |
| 244 | + | + | + | + | + | | + |
| 245 | + | + | + | + | | + | + |
| 246 | + | + | + | | + | + | + |
| 247 | + | + | | + | + | + | + |
| 248 | + | | + | + | + | + | + |
| 249 | + | + | + | + | + | + | + |

+ indicates that the synthetic standard peptide is present whereas the absence of “+” indicates that the synthetic standard peptide is absent.

In one embodiment, the combination does not include the synthetic standard PD-L1 peptide having the amino acid sequence of LQDAGVYR (SEQ ID NO: 15) or NIIQFVHGEEDLK (SEQ ID NO: 16). In this embodiment, the synthetic standard PD-L1 peptide present in the combination could have the amino acid sequence of ILVVDPVTSEHELTCQAEGYPK (SEQ ID NO: 12), LFDVTSTLR (SEQ ID NO: 13), LFNVTSTLR (SEQ ID NO: 14), INTTTNEIFYCTFR (SEQ ID NO: 17) and/or IDTTTNEIFYCTFR (SEQ ID NO: 18), alone or in any combination with each other.

In one embodiment, the combination comprises at least one synthetic standard PD-L-2 peptide. In some additional embodiments, the combination comprises at least two synthetic standard PD-L-2 peptide. In some further embodiments, the combination comprises at least three synthetic standard PD-L-2 peptide. In some additional embodiments, the combination comprises at least four synthetic standard PD-L-2 peptide. In some further embodiments, the combination comprises at least five synthetic standard PD-L-2 peptide. Table 6 below provides some embodiments of the specific PD-L-2 synthetic standard peptides which can be present in the combination of the present disclosure.

TABLE 6
PD-L2 synthetic standard peptides that
could be present in the combination.

| | SEQ ID | SEQ ID | SEQ ID | SEQ ID | SEQ D |
|---|---|---|---|---|---|
| # | NO: 19 | NO: 20 | NO: 21 | NO: 22 | NO: 23 |
| 250 | + | | | | |
| 251 | | + | | | |
| 252 | | | + | | |
| 253 | | | | + | |
| 254 | | | | | + |
| 255 | + | + | | | |
| 256 | | + | + | | |
| 257 | | | + | + | |
| 258 | | | | + | + |
| 259 | + | | + | | |
| 260 | | + | | + | |
| 261 | | | + | | + |
| 262 | + | | | + | |
| 263 | | + | | | + |
| 264 | + | | | | + |
| 265 | + | + | + | | |
| 266 | | + | + | + | |
| 267 | | | + | + | + |
| 268 | + | + | | + | |
| 269 | | + | + | | + |
| 270 | + | + | | | + |
| 271 | + | | + | + | |
| 272 | | + | | + | + |
| 273 | + | | + | | + |
| 274 | + | | | + | + |
| 275 | + | + | + | + | |
| 276 | | + | + | + | + |
| 277 | + | + | + | | + |
| 278 | + | + | | + | + |
| 279 | + | | + | + | + |
| 280 | + | + | + | + | + |

+ indicates that the synthetic standard peptide is present whereas the absence of “+” indicates that the synthetic standard peptide is absent.

In another embodiment, the combination does not include the synthetic standard PD-1L2 peptide having the amino acid sequence of TPEGLYQVTSVLR (SEQ ID NO: 21). In such embodiment, the synthetic standard PD-1L2 peptide present in the combination can have the amino acid sequence of ASFHIPQVQVR (SEQ ID NO: 19), ATLLEEQLPLGK (SEQ ID NO: 20), NFSCVFWNTHVR (SEQ ID NO: 22) and/or DFSCVFWNTHVR (SEQ ID NO: 23), alone or in any combination with each other.

In yet another embodiment, the combination comprises at least one synthetic standard ZAP70 peptide. In some additional embodiments, the combination comprises at least two synthetic standard ZAP70 peptide. In some further embodiments, the combination comprises at least three synthetic standard ZAP70 peptide. Table 7 below provides some embodiments of the specific ZAP70 synthetic standard peptides which can be present in the combination of the present disclosure.

TABLE 7
ZAP70 synthetic standard peptides that
can be present in the combination.

| # | SEQ ID NO: 24 | SEQ ID NO: 25 | SEQ ID NO: 26 |
|---|---|---|---|
| 281 | + | | |
| 282 | | + | |
| 283 | | | + |
| 284 | + | + | |
| 285 | | + | + |
| 286 | + | | + |
| 287 | + | + | + |

+ indicates that the synthetic standard peptide is present whereas the absence of “+” indicates that the synthetic standard peptide is absent.

The synthetic standard peptides of the present disclosure can be obtained synthetically or by recombinant expression in a host cell.

In some embodiments, the synthetic standard peptides can be provided without a label (e.g., label-free or a “light” variant). Label-free synthetic standard peptides can be used in label-free quantitative mass spectrometry, as reagents to prepare a calibration curve or to provide the end user the flexibility of selecting their own label. However, in other embodiments, the synthetic standard peptides can be modified with a label (e.g., a “heavy” variant) that provides a defined mass shift for use during mass spectrometry. In such embodiment, the label can be an isotope, for example a stable isotope. Synthetic standard peptides labeled with a stable isotope can be used as a stable isotope labeled standard (also known as SIS). The stable isotope label can be associated to one or more amino acid residue of the peptide. In an embodiment, the stable isotope label can be associated with one or more arginine residue. SIS synthetic standard peptides can be used to provide a calibration curve as well as to include as internal standards in the sample. In some embodiments, each of the peptides of the combination can be provided in more than one labeled form, each form providing a different mass shift. The differently labeled synthetic standard peptides can be used to provide a multiple-point calibration curve. For example, each of the peptides of the combination can be provided in two distinct labeled forms, each form providing a different mass shift and can be used, for example, to provide a two-point calibration curve.

The synthetic standard peptides of the present disclosure can be labeled using techniques known in the art such as, for example, metabolic or chemical labelling. The synthetic standard peptide can be directly associated with the label or indirectly by introducing one or more linkers between the synthetic standard peptide. The label can be, without limitation, an isotope-coded affinity tag, a dimethyl label, ¹⁸O, or an isobaric tag.

In some embodiments, the synthetic standard peptides (especially those having the amino acid sequence of SEQ ID NO: 11, 14, 17, and 22) may be in a glycosylated form or in a aglycosylated or deglycosylated form, at least in part, before being submitted to mass spectrometry. In some embodiments, it may be advantageous to provide the synthetic standard peptides of the present disclosure in a partially or completely deglycosylated form. In some specific embodiments, it may be necessary to remove one or more glycans that may be present on the peptides. In order to do so, it is possible to submit the synthetic standard peptides to an enzymatic treatment with one or more glycosidases to remove one or more glycans from the synthetic standard peptides. In an embodiment, the N-glycans are removed, at least in part or totally, from the synthetic standard peptides. For example, the glycosidase PNGase F can be used to remove some or all glycans from glycosylated aspargine residues. In an embodiment, the O-glycans are removed, at least in part or totally from the synthetic standard peptides. For example, a neuraminidase (sialidase), β-glcNAcase, β1-4galactosidase, β1-3galactosidase, α-galactosidase, α-fucosidase, α-GalNAcase or a combination thereof can be used to remove some or all glycans from glycosylated serine or threonine residues. In a specific example, the synthetic standard peptide NDSGTYLCGAISLAPK (SEQ ID NO: 11) can be deglycosylated to the synthetic standard peptide DDSGTYLCGAISLAPK (SEQ ID NO: 10), using for example PNGase F, prior to mass spectrometry. In another specific example, the synthetic standard peptide LFNVTSTLR (SEQ ID NO: 14) can be deglycosylated to the synthetic standard peptide LFDVTSTLR (SEQ ID NO: 13), using for example PNGase F, prior to mass spectrometry. In another specific example, the synthetic standard peptide INTTTNEIFYCTFR (SEQ ID NO: 17) can be deglycosylated to the synthetic standard peptide IDTTTNEIFYCTFR (SEQ ID NO: 18), using for example PNGase F, prior to mass spectrometry. In another specific example, the synthetic standard peptide NFSCVFWNTHVR (SEQ ID NO: 22) can be deglycosylated to the synthetic standard peptide DFSCVFWNTHVR (SEQ ID NO: 23), using for example PNGase F, prior to mass spectrometry.

The combination of the synthetic standard peptides of the present disclosure can be provided as a kit which may include instructions on how to use them to quantify the plurality of proteins in the PD-L1 axis in a sample.

### Affinity Agents

In some embodiments, the combination of synthetic standard peptides can be used with a combination of affinity agents to determine, using quantitative mass spectrometry, the expression level of more than one protein in the PD-L1 axis. The affinity agents can be used during sample preparation, prior to mass spectrometry, to positively select the synthetic standard peptides and the corresponding endogenous peptides (e.g., enzymatic fragments which are associated with the proteins in the PD-L1 axis) that may be present in the sample. The combination of affinity agents comprises at least one affinity agent specific for each synthetic standard peptide of the combination. The combination of affinity agents may comprise at least two distinct affinity agents. The combination of affinity agents may comprise at least three distinct affinity agents. The combination of affinity agents may comprise at least four distinct affinity agents. The combination of affinity agents may comprise at least five distinct affinity agents. The combination of affinity agents may comprise at least six distinct affinity agents. The combination may comprise at least seven distinct affinity agents. The affinity agents can be provided on a solid support (such as, for example, a bead or a column) to facilitate the positive selection of the synthetic standard peptides and endogenous peptides associated with proteins in the PD-L1 axis.

Each affinity agent of the present disclosure binds to one synthetic standard peptide as well as to a corresponding endogenous peptide obtained by the proteolytic degradation of a protein in the PD-L1 axis. The binding between the affinity agent and the peptides allows an enrichment of the peptides in the mixture intended to be submitted to quantitative mass spectrometry. In some embodiments, each affinity agent of the present disclosure binds to one synthetic standard peptide as well as to a corresponding endogenous peptide obtained by the proteolytic degradation of a protein in the PD-L1 axis. In the context of the present disclosure, the expressions “specific binding” or “specifically bind” refer to the interaction between two elements in a manner that is determinative of the presence of the elements in the presence or absence of a heterogeneous population of molecules. For example, under designated conditions, the affinity agents of the present disclosure bind to one of the synthetic standard peptide and bind to the synthetic standard/endogenous peptide(s) but not in a specific manner, e.g., they can bind to other peptides as well (such other synthetic standard/endogenous peptides for example).

The combination of affinity agents of the present disclosure include at least two different agents, each agents being specific for a distinct synthetic standard peptide present in the composition. In some embodiments, the combination of affinity agents of the present disclosure can include at least three agents, each agent being specific a distinct synthetic standard peptide present in the composition. In some embodiments, the combination of affinity agents of the present disclosure can include at least four agents, each agent being specific a distinct synthetic standard peptide present in the composition. In some embodiments, the combination of affinity agents of the present disclosure can include at least five agents, each agent being specific a distinct synthetic standard peptide present in the composition. In some embodiments, the combination of affinity agents of the present disclosure can include at least six agents, each agent being specific a distinct synthetic standard peptide present in the composition.

In an embodiment, the affinity agent can be an antibody. In some specific embodiments, the antibody agent can be obtained by immunizing a subject with the synthetic peptide (alone or presented in the form of an immunogen) and obtaining an antibody from the immunized subject. The subject can be, without limitation, a mammal, including a rodent or a camelid. Once obtained from the immunized subject, the antibody can be further modified with methods known in the art. The antibodies of the present disclosure can be polyclonal or monoclonal antibodies as well as single domain antibodies.

The term “antibody”, also referred to as “immunoglobulin” (“Ig”), as used herein refers to a protein comprising at least one heavy or light polypeptide chain. In an embodiment, an antibody comprises a paired heavy and light polypeptide chains. In humans, various Ig isotypes exist, including IgA, IgD, IgE, IgG, and IgM. When an antibody is correctly folded, each chain folds into a number of distinct globular domains joined by more linear polypeptide sequences. For example, the immunoglobulin light chain folds into a variable V_{L} and a constant domain, while the heavy chain folds into a variable V_{H} and three constant domains. Interaction of the heavy and light chain variable domains results in the formation of an antigen binding region (Fv). Each domain has a well-established structure familiar to those of skill in the art.

The light and heavy chain variable regions are responsible for binding the target antigen and can therefore show significant sequence diversity between antibodies. The constant regions show less sequence diversity, and are responsible for binding a number of natural proteins to elicit important biochemical events. The variable region of an antibody contains the antigen-binding determinants of the molecule, and thus determines the specificity of an antibody for its target antigen. The majority of sequence variability occurs in hypervariable regions, three each per variable heavy and light chain; the hypervariable regions combine to form the antigen-binding site, and contribute to binding and recognition of an antigenic determinant. The specificity and affinity of an antibody for its antigen is determined by the structure of the hypervariable regions, as well as their size, shape, and chemistry of the surface they present to the antigen. Various schemes exist for identification of the regions of hypervariability, the two most common being those of Kabat and of Chothia and Lesk. Kabat et al (1991) define the “complementarity-determining regions” (CDR) based on sequence variability at the antigen-binding regions of the V_{H} and V_{L} domains. Chothia and Lesk (1987) define the “hypervariable loops” (H or L) based on the location of the structural loop regions in the V_{H} and V_{L} domains. As these individual schemes define CDR and hypervariable loop regions that are adjacent or overlapping, those of skill in the antibody art often utilize the terms “CDR” and “hypervariable loop” interchangeably, and they may be so used herein. The CDR/loops are referred to herein according to the more recent IMGT numbering system, which was developed to facilitate comparison of variable domains. In this system, conserved amino acids (such as Cys23, Trp41, Cys104, Phe/Trp118, and a hydrophobic residue at position 89) always have the same position. Additionally, a standardized delimitation of the framework regions (FR1: positions 1 to 26; FR2: 39 to 55; FR3: 66 to 104; and FR4: 118 to 129) and of the CDR (CDR1: 27 to 38, CDR2: 56 to 65; and CDR3: 105 to 117) is provided.

A “single domain antibody” or “sdAb” as used herein refers to an antibody that has a single monomeric variable antibody domain comprising at least three complementary determining regions (CDRs). As is known in the art, the single domain antibodies can be obtained from camelids (called also V_{H}H antibodies or nanobodies), from fish (called VNAR antibodies), or by using phage display technology. The single domain antibodies can also be derived from a heavy chain (V_{H}) or a light chain (V_{L}) of an immunoglobulin. Examples of single domain antibodies of the present disclosure include but are not limited to V_{H}H or nanobodies. Single domain antibodies may be further defined by a size of less than about 15 kDa, less than about 14 kDa, less than about 13 kDa, less than about 12 kDa, less than about 11 kDa, or less than about 10 kDa. In one embodiment, sdAbs or nanobodies can be defined as having less than about 150 amino acids, less than about 140 amino acids, less than about 130 amino acids, less than about 120 amino acids, less than about 110 amino acids or less than 100 amino acids. In a further embodiment, nanobodies can be defined as consisting of less than about 150 amino acids, less than about 140 amino acids, less than about 130 amino acids, less than about 120 amino acids, less than about 110 amino acids or less than 100 amino acids.

The sdAb of the present disclosure may be derived from naturally-occurring sources. Heavy chain antibodies of camelid origin lack light chains and thus their antigen binding sites consist of one domain, termed V_{H}H. sdAbs have also been observed in shark and are termed VNAR. Other sdAb may be engineered based on human Ig heavy and light chain sequences. The term “sdAb” may include those sdAbs directly isolated from VHH or VNAR, those synthetically prepared from human light or heavy chains, and those obtained from phage display or other technologies. The sdAbs can be derived from the aforementioned sdAbs, recombinantly produced sdAbs, as well as those sdAbs generated through affinity maturation, stabilization, solubilization, camelization, or other methods of antibody engineering.

Because the synthetic standard peptides of the present disclosure include an epitope which is also present on a corresponding protein or endogenous peptide of a protein in the PD-L1 axis, the synthetic standard peptides can be used in the process of obtaining or maturing the antibodies of the present disclosure. The synthetic standard peptides may be administered to a subject (such as a mammal or a camelid) to elicit the production of antibodies specific against the corresponding protein in the PD-L1 axis. The synthetic standard peptides may be presented in the form of an immunogen, a chemical entity which includes the synthetic standard peptide and that is recognized by the immune system to trigger antibody production. The synthetic standard peptides may be used to select antibodies presented on phages which possess affinity and specificity towards the corresponding protein in the PD-L1 axis. In such application, the synthetic standard peptides may be used in an isolated form or as an immunogen (which can be in a chimeric form with a carrier).

When antibodies are used as affinity agents, they can be provided on a solid support to facilitate the positive selection and enrichment of the synthetic standard peptides and endogenous peptides derived from the proteins in the PD-L1 axis. In some embodiments, the antibodies can be covalently associated with the solid support. Furthermore, when associating the antibody with a solid support, care should be taken to maintain the antibody's ability to recognized its target (e.g., the epitope present on the synthetic standard peptide and the corresponding endogenous peptide derived from a protein in the PD-L1 axis).

In another embodiment, the affinity agent can be an aptamer. The aptamers of the present disclosure are single-stranded molecules composed of deoxyribonucleic acid (DNA) nucleotides, ribonucleic acid (RNA) nucleotides or a combination of both deoxyribonucleic and ribonucleic acid nucleotides. In an embodiment, the aptamers of the present disclosure are exclusively made of deoxyribonucleic acid (DNA) nucleotides. The aptamers can be composed of naturally-occurring nucleobases (also referred to as bases), sugars and covalent internucleoside (backbone) linkages. The aptamers can also have “non-naturally-occurring” or “synthetic” portions which function similarly. In the context of the present disclosure, the term “nucleotides” refers to a deoxyribonucleic acid nucleotide or to a ribonucleic acid nucleotides.

The aptamers of the present disclosure can include various modifications, e.g., stabilizing modifications, and thus can include at least one modification in the phosphodiester linkage and/or on the sugar, and/or on the base. For example, the aptamer can include one or more phosphorothioate linkages, phosphorodithioate linkages, and/or methylphosphonate linkages. Different chemically compatible modified linkages can be combined, e.g., modifications where the synthesis conditions are chemically compatible. While modified linkages are useful, the aptamer can include phosphodiester linkages, e.g., include at least one phosphodiester linkage, or at least 5%, 10%, 20%, 30% or more phosphodiester linkages. Additional useful modifications include, without restriction, modifications at the 2′-position of the sugar (such as 2′-O-alkyl modifications, 2′-O-methyl modifications, 2′-amino modifications, 2′-halo modifications (e.g., 2′-fluoro) as well as acyclic nucleotide analogs. In another embodiment, the aptamer has modified linkages throughout, e.g., phosphorothioate; has a 3′- and/or 5′-cap; includes a terminal 3′-5′ linkage.

In some embodiments, the aptamer includes a concatemer and comprises two or more oligonucleotide sequences joined by one or more linker. The linker may, for example, consist of modified nucleotides or non-nucleotide units. In some embodiments, the linker can provide flexibility to the aptamer. The use of concatemers as aptamers can provide a facile method to synthesize a final molecule, by joining smaller oligonucleotides building blocks to obtain the desired length. For example, a 12 carbon linker (C₁₂ phosphoramidite) can be used to join two or more concatemers and provide length, stability and flexibility.

The aptamers of the present disclosure can include a natural or a non-natural backbone. Non-natural or synthetic backbones include, for example, phosphorothioates, chiral phosphorothioates, phosphorodithioates, phosphotriesters aminoalkylphosphotri-esters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, carboranyl phosphate and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Aptamers having inverted polarity typically include a single 3′ to 3′ linkage at the 3′-most internucleotide linkage i.e. a single inverted nucleoside residue which may be abasic (the nucleobase is missing or has a hydroxyl group in place thereof). The backbones of some exemplary modified aptamers that do not include a phosphodiester linkage have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S, and CH₂ component parts. Particularly advantageous are backbone linkages that include one or more charged functional groups.

The aptamers of the present disclosure may also contain one or more substituted sugar moieties. For example, such oligonucleotides can include one of the following 2′-modifications: OH; F; O—, S—, or N-alkyl; O—, S—, or N-alkenyl; O-, S- or N-alkynyl; or O-alkyl-O-alkyl, wherein the alkyl, alkenyl and alkynyl may be substituted or unsubstituted C1 to C₁₀ alkyl or C₂ to C₁₀ alkenyl and alkynyl, or 2′-O—(O-carboran-1-yl)methyl. Particular examples are O[(CH₂)ₙO]ₘCH₃, O(CH₂)˜OCH₃, O(CH₂)ₙNH₂, O(CH₂)ₙCH₃, O(CH₂)ₙONH₂, and O(CH₂)ₙON [(CH₂)ₙCH₃)]₂, where n and m are from 1 to 10. Other exemplary aptamers can include one of the following 2′-modifications: C, to Cao lower alkyl, substituted lower alkyl, alkenyl, alkynyl, alkaryl, aralkyl, O-alkaryl or O-aralkyl, SH, SCH₃, OCN, Cl, Br, CN, CF₃. OCF₃, SOCH₃, SO₂CH₃, ONO₂, NO₂, N₃, NH₂, heterocycloalkyl, heterocycloalkaryl, aminoalkylamino, polyalkylamino or substituted silyl.

Other modifications to the aptamers include Locked Nucleic Acids (LNAs) in which the 2′-hydroxyl group is linked to the 3′ or 4′ carbon atom of the sugar ring thereby forming a bicyclic sugar moiety. Other modifications include 2′-methoxy (2′-O—CH₃), 2′-methoxyethyl (2′O—CH₂—CH₃), 2′-ethyl, 2′-ethoxy, 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F).

The 2′-modification may be in the arabino (up) position or ribo (down) position. Similar modifications may also be made at other positions on the aptamers, particularly the 3′ position of the sugar on the 3′ terminal nucleotide or in 2′-5′ linked oligonucleotides and the 5′ position of the 5′ terminal nucleotide. The aptamers may also have sugar mimetics such as cyclobutyl moieties in place of the pentofuranosyl sugar.

The aptamers of the present disclosure can include “unmodified” or “natural” bases (nucleobases) such as adenine (A) and guanine (G), and the pyrimidine bases thymine (T), cytosine (C) and uracil (U). The aptamers may also include base modifications or substitutions. Modified bases include, but are not limited to other synthetic and naturally-occurring bases such as 5-methylcytosine, 5-hydroxymethyl cytosine, xanthine, hypoxanthine, 2-aminoadenine, 6-methyl, and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and 3-deazaguanine and 3-deazaadenine. Modified bases may also include those in which the purine or pyrimidine base is replaced with other heterocycles, for example 7-deaza-adenine, 7-deazaguanosine, 2-aminopyridine and 2-pyridone.

Another type of modification that can be included in the aptamers of the present disclosure are phosphorodithioate linkages. The aptamers comprising modified oligonucleotides containing phosphorothioate or dithioate linkages may also contain one or more substituted sugar moieties particularly modifications at the sugar moieties including, without restriction, 2′-ethyl, 2′-ethoxy, 2′-methoxy, 2′-aminopropoxy, 2′-allyl, 2′-fluoro, 2′-pentyl, 2′-propyl, 2′-dimethylaminooxyethoxy, and 2′-dimethylaminoethoxyethoxy. The 2′-modification may be in the arabino (up) position or ribo (down) position. A preferred 2′-arabino modification is 2′-fluoro. Similar modifications may also be made at other positions on the aptamer, particularly the 3′ position of the sugar on the 3′ terminal nucleotide or in 2′-5′ linked oligonucleotides and the 5′ position of 5′ terminal nucleotide.

In a further embodiment, the affinity agent can be a microparticle. The microparticles of the present disclosure are particles with a diameter of 1-1000 μm. The microparticle can bind subsets of proteins and peptides (referred to as protein corona) in a mixture based on their specific chemical characteristics. Therefore, the microparticles can be used to specifically enrich for one or more protein in the PD-L1 axis, the synthetic standard proteins and/or the endogous proteins. After release of the bound proteins/peptides, these can be analyzed by mass spectrometry. In some embodiments, the microparticles can be made of polymers, glass, or ceramics. In additional embodiments, the microparticles can have different structures, being either spherical or non-spherical: mononuclear/single core/core-shell, multi-wall, polynuclear/multiple core, matrix, coated polynuclear core, coated matrix particle, patchy microparticle, dual-compartment microcapsule, colloidosome, giant liposome, irregular-shaped microparticle, torus-shaped microparticle, bullet-shaped microparticle, microtablet, and cubic-shaped microparticle.

The combination of affinity agents of the present disclosure can be provided as a kit which may include instructions on how to use them to quantify the plurality of proteins in the PD-L1 axis in a sample.

### Methods for Quantifying a Plurality of Proteins in the PD-L1 Axis

The combination of synthetic standard peptides is intended to be used to quantify the amount of a plurality of proteins in the PD-L1 axis in a sample using quantitative mass spectrometry. The method can be used on various types of biological samples comprising cells suspected of having more than one proteins in the PD-L1 axis. The sample is obtained or derived from a human subject. The sample can, in some embodiments, comprise at least one cell or part of a cell suspected of having expressed one or more protein in the PD-L1 axis. The cell or part of the cell can be obtained from an in vitro, ex vivo or in vivo source. In some specific embodiment, the cell can be obtained from a patient's derived xenograft. The cell or part of the call can be suspected of being cancerous. In some embodiments, the sample comprises a fluid, a cell, a tissue or a combination thereof. In some further embodiments, the sample comprises a living cell, a frozen cell or a fixed cell. In some additional embodiments, the sample comprises a living tissue, a frozen tissue, or a fixed tissue.

The quantitative mass spectrometry methods of the present disclosure endogenous peptides derived from the proteins in the PD-L1 axis to determine the amount (or level of expression) of the proteins in the PD-L1 axis. In the methods of the present disclosure, the amount of at least two proteins in the PD-L1 axis is determined. As such, the method can be used to determine the amount of any two of the following proteins: LCK, NT5E, PD-1, PD-L1, PD-L2 and ZAP70. In some embodiments, the methods of the present disclosure, the amount of at least three proteins in the PD-L1 axis is determined. As such, the method can be used to determine the amount of any three of the following proteins: LCK, NT5E, PD-1, PD-L1, PD-L2 and ZAP70. In some embodiments, the methods of the present disclosure, the amount of at least four proteins in the PD-L1 axis is determined. As such, the method can be used to determine the amount of any four of the following proteins: LCK, NT5E, PD-1, PD-L1, PD-L2 and ZAP70. In some embodiments, the methods of the present disclosure, the amount of at least five proteins in the PD-L1 axis is determined. As such, the method can be used to determine the amount of any five of the following proteins: LCK, NT5E, PD-1, PD-L1, PD-L2 and ZAP70. In some embodiments, the methods of the present disclosure, the amount in all six proteins in the PD-L1 axis is determined. As such, the method can be used to determine the amount of all of the following proteins: LCK, NT5E, PD-1, PD-L1, PD-L2 and ZAP70.

In a first step of the method of the present disclosure, a treated biological sample suspected of comprising endogenous peptides derived from at least two proteins in the PD-L1 axis is obtained. In some embodiments, the biological sample can be chemically treated (with cyanogen bromide for example) to obtain the endogenous peptides. In another embodiment, the biological sample can be submitted to an enzymatic (proteolytic) treatment to obtain the endogenous peptides. As such, the endogenous peptides can be obtained by contacting the proteins of the sample with one or more proteolytic enzymes which includes, but is not limited to, trypsin, chymotrypsin, endoproteinase Asp-N, endoproteinase Lys-C, endoproteinase Glu-C, and/or subtilisin. In a specific embodiment, the endogenous peptides can be obtained by contacting the proteins of the sample with trypsin. In some embodiments, the method includes enzymatically treating the proteins of the biological sample to obtain the endogenous peptides.

In some embodiments, it may be advantageous to provide the endogenous peptides in a partially or completely deglycosylated form. In some specific embodiments, it may be necessary to remove one or more glycans that may be present on the endogenous peptides. In order to do so, it is possible to submit the endogenous peptides to an enzymatic treatment with one or more glycosidases to remove one or more glycans from the synthetic standard peptides. In an embodiment, the N-glycans are removed, at least in part or totally, from the endogenous peptides. For example, the glycosidase PNGase F can be used to remove some or all glycans from glycosylated aspargine residues. In an embodiment, the O-glycans are removed, at least in part or totally from the endogenous peptides. For example, a neuraminidase (sialidase), β-glcNAcase, β1-4galactosidase, β1-3galactosidase, α-galactosidase, α-fucosidase, α-GalNAcase or a combination thereof can be used to remove some or all glycans from glycosylated serine or threonine residues.

In some embodiments, the method can comprise obtaining the biological sample from a cell or a subject and subsequently treating it to submit it to quantitative mass spectrometry. The method can include, in some embodiments, obtaining a biological sample. For example, the method can optionally include culturing in vitro or expanding in vivo (to provide a xenograft for example) the cells of the biological sample. In another example, the method can include, in some embodiments, freezing or fixing the biological sample (prior to or after the protein extraction step) according to techniques known in the art. In some additional embodiments, the method can comprises using fixed (and optionally stained) biological samples. As such, in a specific embodiment, the method may comprise fixing (and optionally staining) the biological sample with formaldehyde or a derivative thereof.

In another embodiment, the method can include extracting the proteins from such biological sample. The protein extraction can be performed using depraffinization, acid precipitation, organic solvent precipitation, extraction with detergents or aqueous and organic solvent mixtures, etc. Protein extraction may also include methods of cell and tissue disruption, including sonication, adaptive focused acoustics, cryopulverization, mechanical disruption with beads, and extrusion through membranes. Proteins may be extracted using techniques that yield total protein samples representative of all proteins present in the biological sample.

Once the endogenous peptides have been obtained, they are combined with the combination of synthetic standard peptides of the present disclosure to provide a supplemented mixture. The type of synthetic standard peptides that is combined with the endogenous peptides will depend on the protein in the PD-L1 axis that is being quantified by the method. It will be necessary to include at least one synthetic standard peptide for each protein in the PD-L1 axis that is being quantified. If the method seeks at quantifying the amount of LCK in the sample, the combination of synthetic standard peptides will necessarily include a synthetic standard LCK peptide. If the method seeks at quantifying the amount of NT5E in the sample, the combination of synthetic standard peptides will necessarily include a synthetic standard NT5E peptide. If the method seeks at quantifying the amount of PD-1 in the sample, the combination of synthetic standard peptides will necessarily include a synthetic standard PD-1 peptide. If the method seeks at quantifying the amount of PD-L1 in the sample, the combination of synthetic standard peptides will necessarily include a synthetic standard PD-L1 peptide. If the method seeks at quantifying the amount of PD-L2 in the sample, the combination of synthetic standard peptides will necessarily include a synthetic standard PD-L2 peptide. If the method seeks at quantifying the amount of ZAP70 in the sample, the combination of synthetic standard peptides will necessarily include a synthetic standard ZAP70 peptide. The glycosylation that may be present on the synthetic standard peptide or the endogenous peptide can be removed prior to the enzymatic digestion or after.

In some embodiments, a labeled heavy version (e.g., a SIS variant) of the synthetic standard peptide is contacted with the enzymatically-treated sample. In additional embodiment, a first labeled version (e.g., a SIS) and a second labeled version (e.g., a dSIS—having 2 a different label than the SIS) of the same synthetic standard peptide can be contacted with the enzymatically-treated sample. As it is known in the art, SIS and dSIS can be spiked at different concentrations in the supplemented mixture, the concentrations typically in the range of amol to fmol of peptide per μg of total lysate protein. It will be recognized that the concentration of SIS and dSIS can vary, depending on the reference concentration ranges of the individual proteins in the PD-L1 axis of the sample.

Once obtained, the supplemented mixture is enriched for the synthetic standard peptides and their related endogenous peptides by using the affinity agents described herein to obtain an enriched mixture. Due to their binding specificity, the affinity agents are able to bind and positively select, from the supplemented mixture, the synthetic standard peptides and the endogenous peptides derived from one or more of the proteins in the PD-L1 axis. It will be necessary to include at least one affinity agent for each protein in the PD-L1 axis that is being quantified. If the method seeks at quantifying the amount of LCK in the sample, the combination of affinity agents will necessarily include an affinity agent specific for LCK and its related synthetic standard LCK peptide. If the method seeks at quantifying the amount of NT5E in the sample, the combination of affinity agents will necessarily include an affinity agent specific for NT5E and its related synthetic standard NT5E peptide. If the method seeks at quantifying the amount of PD-1 in the sample, the combination of affinity agents will necessarily include an affinity agent specific for PD-1 and its associated synthetic standard PD-1 peptide. If the method seeks at quantifying the amount of PD-L1 in the sample, the combination of affinity agents will necessarily include an affinity agent specific for PD-L1 and its associated synthetic standard PD-L1 peptide. If the method seeks at quantifying the amount of PD-L2 in the sample, the combination of affinity agents will necessarily include an affinity agent specific for PD-L2 and its associated synthetic standard PD-L2 peptide. If the method seeks at quantifying the amount of ZAP70 in the sample, the combination affinity agents will necessarily include an affinity agent specific for ZAP70 as well as it associated synthetic standard ZAP70 peptide.

Once the enriched mixture has been obtained, it is submitted to quantitative mass spectrometry to determine the abundance (or amount/concentration) of the plurality of the proteins in the PD-L1 axis. The quantitative mass spectrometry can be, for example, multiple reaction monitoring mass spectrometry, parallel reaction monitoring mass spectrometry, or matrix-assisted laser desorption/ionization (MALDI) mass spectrometry. In one embodiment, the quantitative mass spectrometry is MALDI mass spectrometry (such as, for example an immuno-multiple reaction monitoring mass spectrometry). Because of the specific enrichment of the synthetic standard peptides and their related endogenous peptides by the affinity agents, the enriched sample is of low complexity and can be directly analyzed by MALDI mass spectrometry (instead of LC-MS). This strategy is not applicable without prior affinity enrichment by the affinity agents, as the lack of affinity enrichment will require either extensive fractionation (time, cost, not reproducible, not robust) or extremely sensitive instrumentation that are not compatible with clinical standards (very expensive, high maintenance, very prone to errors and downtimes). The advantage of the MALDI-based strategy are, that it does not require an LC system that is typically associated with (high) maintenance requirements and that also reduces the robustness of the clinical assay. LC-MS also requires considerably more time for analysis, from 5-60 min per sample, while MALDI can be processed in a fully automated manner, measuring multiple samples per minute.

In some embodiments, the method of the present disclosure can include comparing the results obtained to a calibration curve to make the determination in abundance in the plurality of proteins in the PD-L1 axis. In some additional embodiments, the method of the present disclosure can include acquiring a calibration curve from at least one or a plurality of synthetic standard peptides disclosed herein. The calibration curve can be a single-point calibration curve. In some embodiments, the calibration curve can comprise at least one or more point. The calibration curve can be a two-point calibration curve. In some embodiments, the calibration curve can comprise at least two or more points. The calibration curve can be a three-point calibration curve. In some embodiments, the calibration curve can comprise at least three or more points. The calibration curve can be a four-point calibration curve. In some embodiments, the calibration curve can comprise at least four or more points. The calibration curve can be a five-point calibration curve. In some embodiments, the calibration curve can comprise at least five or more points. The calibration curve can be a six-point calibration curve. In some embodiments, the calibration curve can comprise at least six or more points. The calibration curve can be a seven-point calibration curve. In some embodiments, the calibration curve can comprise at least seven or more points. The calibration curve can be an eight-point calibration curve. In some embodiments, the calibration curve can comprise at least eight or more points. The calibration curve can be a nine-point calibration curve. In some embodiments, the calibration curve can comprise at least nine or more points. The calibration curve can be a ten-point calibration curve. In some embodiments, the calibration curve can comprise at least ten or more points. In some embodiments the method of the present disclosure can include comparing the results obtained to an internal calibration curve generated through adding 2 or more different SIS standards to the sample at different concentrations, in order to make the determination in abundance in the plurality of proteins in the PD-L1 axis as disclosed in Ibrahim et al., 2020.

### Therapeutic Uses

The method for quantifying a plurality of proteins in the PD-L1 axis as disclosed herein can be useful to predict the response of a subject to an immune checkpoint inhibitor and/or to an immune checkpoint activating agent (collectively referred to herein as an “immune checkpoint modulating agent”). In some embodiments, the present disclosure comprises first determining if the expression of one or more proteins in the PD-L1 axis is modulated in a sample of a subject to determine if it would be useful for a cell or a subject to be contacted with or receive an immune checkpoint modulating agent. In additional embodiments, the present disclosure can comprise contacting the cell with or administering the subject with the immune checkpoint modulating agent if it has been determined that the cell or subject exhibits a modulation in the expression of one or more proteins in the PD-L1 axis. In further embodiments, the present disclosure can comprise determining the expression of one or more proteins in the PD-L1 axis have been modulated after the cell has been contacted with or the after subject has received at least one dose of an immune checkpoint modulating agent to determine if the immune checkpoint modulating agent did exhibit effects in the cell or the subject. The methods of the present disclosure can be applied to any cells, including but not limited to a mammalian cell, such as a human cell. The therapeutic uses of the present disclosure can be applied to any subjects, including but not limited to mammals, such as a human subject.

As used in the context of the present disclosure, the expression “immune checkpoint modulating agent” refers to a therapeutic agent or a combination of therapeutic agents capable of increasing the biological activity of immune response cells (lymphocytes such as, for example, B cells and T cells, macrophages, dendritic cells, natural killer cells, neutrophils, etc.) for stimulating the immune response against a cancer cell (which may be, in some embodiments, a metastatic cancer cell). In some embodiments, the increase in biological activity is observed in tumor-associated immune cells. This increase in biological activity does not have to be permanent, it can be transient. The cancer cell can be in vitro or in vivo.

The cancer can be, for example, a lung cancer (such as, for example, a non-small-cell lung cancer or a small-cell cancer). The cancer can be, for example, a glioma. The cancer can be, for example, a breast cancer, a liver cancer (such as, for example, an hepatocellular carcinoma), a kidney/renal cancer (such as, for example, a renal cell carcinoma), a stomach cancer, a colorectal cancer, a head and neck tumor, an ovarian cancer, a bladder cancer, a skin cancer (such as, for example, a squamous cell carcinoma, a basal cell carcinoma, a Merkel cell carcinoma, a cutaneous melanoma or a uveal melanoma), an esophagus cancer, a fallopian tube cancer, a genitourinary tract cancer (such as, for example, a transitional cell carcinoma or an endometrioid carcinoma), a prostate cancer (such as, for example, a hormone refractory prostate cancer), a stomach cancer, a nasopharyngeal cancer (such as, for example, a nasopharyngeal carcinoma), a peritoneal cancer, an adrenal gland cancer, an anal cancer, a thyroid cancer (such as, for example, an anaplastic thyroid cancer), a biliary cancer (such as, for example, cholangiocarcinoma), a gastro-intestinal cancer, a mouth cancer, a nervous system cancer, a penis tumor and/or a thymic cancer. The cancer can be a melanoma, a sarcoma, a mesothelioma, a glioblastoma, a lymphoma (such as, for example, a B-cell lymphoma (including diffuse large B-cell lymphoma), Hodgkins disease, a non-Hodgkin lymphoma, a multiple myeloma, a follicle center lymphoma, a peripheral T-cell lymphoma, a primary mediastinal large B-cell lymphoma or a myelodysplastic syndrome), a leukemia (such as, for example, an acute myelogenous leukemia, a chronic lymphocytic leukemia or a chronic myelocytic leukemia), a glioma and/or a melanoma. The cancer can be a stage I cancer, a stage II cancer, a stage Ill cancer, or a stage VII cancer. The cancer can be a metastatic cancer. The cancer can be a hormone-sensitive or a hormone-refractory cancer.

In some embodiments, the method can include determining the expression of more than one protein in the PD-L1 axis in the subject intended to receive the immune checkpoint modulating agent or having received at least one dose of immune checkpoint modulating agent. This determination step can be done to determine if additional doses of the immune checkpoint modulating agent should be administered to the subject.

The determination can be made on two, three, four, five, or six proteins in the PD-L1 axis (before or after the administration of the immune checkpoint modulation agents). In some embodiments, an expression level of a protein in the PD-L1 axis is considered to be modulated when it is either increased or decreased with respect to a control expression level (obtained for example from a healthy control cell or a healthy control subject). In a specific embodiment, the increase in the expression of at least two proteins in the PD-L1 axis is indicative that an immune checkpoint modulating agent can or should be used in the cell or the subject to reduce the biological activity of at least two proteins in the PD-L1 axis. In a specific embodiment, the decrease in the expression of at least two proteins in the PD-L1 axis is indicative that an immune checkpoint modulating agent can or should be used in the cell or the subject to increase the biological activity of at least two proteins in the PD-L1 axis.

In an embodiment, the immune checkpoint modulating agent is a small molecule. In another embodiment, the immune checkpoint modulating agent is an antibody or an antibody derivative which is specific for an immune cell (and in some embodiments, a polypeptide which is associated with a protein expressed on the surface of an immune cell or a ligand recognized by a protein expressed on the surface of an immune cell). The antibodies are specific for at least one antigen. As used in the context of the present disclosure, an antibody is “specific for an antigen” when it is able to discriminate specifically the antigen from other (related or unrelated antigens). In some embodiments, the antigen is present on various immune cells and as such, even though the antibody is specific for an antigen, it can bind with specificity to different immune cells. The antibodies disclosed herein can be polyclonal or monoclonal antibodies. The antibodies can be antibody derivatives, such as, for example, a chimeric antibody or a humanized antibody. The antibodies can be single domain antibodies, including but not limited to, nanobodies.

In still further embodiments, the immune checkpoint modulating agent can be an antagonistic antibody. The expression “antagonistic antibody” refers to an antibody capable of reducing (e.g., decreasing, inhibiting, or abrogating) the biological activity of a receptor present on an immune cell. In some specific embodiments, the immune checkpoint modulating agent can be antagonistic or agonistic to a specific immune checkpoint.

In such embodiment, the immune checkpoint modulating agent can be an antagonistic antibody for the immune checkpoint or a neutralizing antibody to the ligand of the immune check-point. For example, the immune checkpoint can be the cytotoxic T-lymphocyte associated protein 4 (CTLA-4) protein and the immune checkpoint modulating agent can be an anti-CTLA-4 antibody (such as ipilimumab or tremelimumab) or an anti-CTLA-4 ligand antibody. In another embodiment, the immune checkpoint can be the programmed cell death 1 (PD-1) protein and the immune checkpoint modulating agent can be an anti-PD-1 antibody (such as, for example, pembrolizumab or nivolumab, pembrolizumab or pidilizumab), an anti-PD-1 ligand antibody (e.g., an anti-programmed death ligand 1 (PD-L1) antibody, such as, for example atezolizumab, avelumab or durvalumab), or anti-PD-L2 ligand antibodies (e.g., an anti-programmed death ligand 2 (PD-L2) antibody). In still another example, the immune checkpoint can be the T-cell immunoglobulin and mucin-domain containing-3 (TIM-3) protein and the immune checkpoint modulating agent can be an anti-TIM-3 antibody or an anti-TIM-3 ligand antibody. In yet another example, the immune checkpoint can be the lymphocyte-activation gene 3 (LAG-3) protein and the immune checkpoint modulating agent can be an anti-LAG-3 antibody or an anti-LAG-3 ligand antibody. In another example, the immune checkpoint can be CD244 (also referred to as 2B4) and the immune checkpoint modulating agent can be an anti-CD244 antibody or an anti-CD244 ligand antibody. In a further example, the immune checkpoint can be the T cell immunoreceptor with Ig and ITIM domains (TIGIT) protein and the immune checkpoint modulating agent can be an anti-TIGIT antibody or an anti-TIGIT ligand antibody (such as, for example, an anti-CD155 antibody). In a further example, the immune checkpoint can be CD96 and the immune checkpoint modulating agent can be an anti-CD96 antibody or an anti-CD96 ligand antibody (such as, for example, an anti-CD155 antibody). In still another example, the immune checkpoint can be V-domain Ig suppressor of T cell activation (VISTA) protein and the immune checkpoint modulating agent can be an anti-VISTA antibody or an anti-VISTA ligand antibody. In yet another example, the immune checkpoint can be CD112R and the immune checkpoint modulating agent can be an anti-CD112R antibody or an anti-CD112R ligand (such as, for example, an anti-CD112 antibody).

In the methods of the present disclosure, it is contemplated to consider a cell or subject as being responsive towards an immune checkpoint modulating agent if the expression level of more than one protein in the PD-L1 axis has been determined to be modulated in the cell or in the subject. The modulation can be observed by comparing, for example, the level of expression of more than one protein in the PD-L1 axis with a control level of expression (obtained from or derived from a control (healthy) cell or a control (healthy) subject).

In some embodiments, a cell or a subject is considered responsive to an immune checkpoint modulating agent specific for PD-1 if the expression level of PD-1 and at least one more protein in the PD-L1 axis has been determined to be modulated in the cell or in the subject. Alternatively or in combination, it is contemplated to contact a cell or administer the subject with an immune checkpoint modulating agent specific for PD-1 if the expression level of PD-1 and at least one more protein in the PD-L1 axis has been determined to be modulated in the cell or in the subject. In some embodiments, it is contemplated to contact a cell with or administer the subject with an immune checkpoint modulating agent specific for PD-1 if the expression level of PD-L1 and at least one more protein in the PD-L1 axis has been determined to be increased in the cell or in the subject.

In some embodiments, a cell or a subject is considered responsive to an immune checkpoint modulating agent specific for PD-L1 if the expression level of PD-L1 and at least one more one protein in the PD-L1 axis has been determined to be modulated (and in some embodiments increased) in the cell or in the subject. Alternatively or in combination, it is contemplated to contact a cell or administer the subject with an immune checkpoint modulating agent specific for PD-L1 if the expression level of PD-L1 and at least one more protein in the PD-L1 axis has been determined to be modulated in the cell or in the subject. In some embodiments, it is contemplated to contact a cell with or administer the subject with an immune checkpoint modulating agent specific for PD-L1 if the expression level of PD-L1 and at least one more protein in the PD-L1 axis has been determined to be increased in the cell or in the subject.

In some embodiments, a cell or a subject is considered responsive to an immune checkpoint modulating agent specific for PD-L2 if the expression level of PD-L2 and at least one more protein in the PD-L1 axis has been determined to be modulated (and in some embodiments increased) in the cell or in the subject. Alternatively or in combination, it is contemplated to contact a cell with or administer the subject with an immune checkpoint modulating agent specific for PD-L2 if the expression level of PD-L2 and at least one more protein in the PD-L1 axis has been determined to be modulated in the cell or in the subject. In some embodiments, it is contemplated to contact a cell with or administer the subject with an immune checkpoint modulating agent specific for PD-L2 if the expression level of PD-L2 and at least one more protein in the PD-L1 axis has been determined to be increased in the cell or in the subject.

In another embodiment, the immune checkpoint modulating agent can be an agonistic antibody. The expression “agonistic antibody” refers to an antibody capable of upregulating (e.g., increasing, potentiating or supplementing) the biological activity of a cell-surface receptor to ultimately upregulate the biological activity of the immune cell expressing such receptor and stimulate the immune response. In an example, the receptor can be a TNF receptor superfamily member 4 protein (referred to as TNFRSF4 or OX40) and the immune checkpoint modulating agent can be an anti-TNFRSF4 antibody. In still another example, the receptor can be a TNF receptor superfamily member 9 protein (referred to as TNFRSF9 or CD137 or 41BB) and the immune checkpoint modulating agent can be an anti-TNFRSF9 antibody. In still another example, the receptor can be a TNF receptor superfamily member 18 protein (referred to as TNFRSF18 or GITR) and the immune checkpoint modulating agent can be an anti-TNFRSF18 antibody. In still another example, the receptor can be a CD27 protein and the immune checkpoint modulating agent can be an anti-CD27 antibody. In still another example, the receptor can be a CD28 protein and the immune response checkpoint inhibitor agent can be an anti-CD28 antibody. In yet another example, the receptor can be a CD40 protein and the immune checkpoint modulating agent can be an anti-CD40 antibody.

In another embodiment, the immune checkpoint modulating agent can be an antagonistic antibody for a cell-surface protein (including a cell receptor) or a soluble protein (including a cell receptor ligand) expressed by tumor cells and/or immune cells whose activity need to be reduced, abolished or inhibited to increase the biological activity of immune cell against tumor cells. In an example, the cell-surface protein can be a CD47 protein and the immune checkpoint modulating agent can be an anti-CD47 antibody. In another example, the cell-surface protein can be a macrophage colony-stimulating factor receptor (M-CSFR also known as CSF1R) and the immune checkpoint modulating agent can be an anti-M-CSFR antibody. In still another example, the cell-surface and soluble protein can be NT5E/CD73 and the immune checkpoint modulating agent can be an anti-NT5E/CD73 antibody. In yet another example, the cell-surface protein can be CD39 and the immune checkpoint modulating agent can be an anti-CD39 antibody. In yet another example, the cell-surface protein can be C—C chemokine receptor type 2 (CCR2 or CD192) and the immune checkpoint modulating agent can be an anti-CCR2 antibody. In yet another example, the cell-surface protein can be C—C chemokine receptor type 2 (CCR2 or CD192) and the immune-checkpoint-modulating agent can be an anti-CCR2 antibody. In yet another example, the cell-surface protein can be C—C chemokine receptor type 2 (CCR2 or CD192) and the immune checkpoint modulating agent can be an anti-CCR2 antibody. In yet another example, the cell-surface protein can be a mannose receptor (CD206) and the immune checkpoint modulating agent can be an anti-CD206 antibody. In yet another example, the cell-surface protein can be CD-163 and the immune checkpoint modulating agent can be an anti-CD163 antibody. In still another example, the soluble protein can be CCL2 and the immune checkpoint modulating agent can be an anti-CCL2 antibody. In still a further example, the soluble protein can be transforming growth factor β (TGFβ) and the immune checkpoint modulating agent can be an anti-TGFβ antibody. In yet a further embodiment, the soluble protein can be interleukin-10 (IL-10) and the immune checkpoint modulating agent can be an anti-IL-10 antibody. In still a further embodiment, the soluble protein can be interleukin-6 (IL-6) and the immune checkpoint stimulating agent can be an anti-IL-6 antibody. In another embodiment, the soluble protein can be the vascular endothelial growth factor (VEGF) and the immune checkpoint modulating agent can be an anti-VEGF antibody. In yet another example, the soluble protein can be a chemokine ligand 1 (CXCL1) and the immune checkpoint modulating agent can be an anti-CXCL1 antibody. In yet another example, the soluble protein can be a chemokine ligand 2 (CXCL2) and the immune checkpoint modulating agent can be an anti-CXCL2 antibody. In yet another example, the soluble protein can be arginase 1 (ARG1) and the immune checkpoint modulating agent can be an anti-ARG1 antibody.

In some embodiments, a cell or a subject is considered responsive to an immune checkpoint modulating agent specific for NT5E/CD73 if the expression level of NT5E/CD73 and at least one additional protein in the PD-L1 axis has been determined to be modulated (and in some embodiments increased) in the cell or in the subject. Alternatively or in combination, it is contemplated to contact a cell or administer the subject with an immune checkpoint modulating agent specific for NT5E/CD73 if the expression level of NT5E/CD73 and at least one two additional protein in the PD-L1 axis has been determined to be modulated in the cell or in the subject. In some embodiments, it is contemplated to contact a cell or administer the subject with an immune checkpoint modulating agent specific for NT5E/CD73 if the expression level NT5E/CD73 and at least three additional protein in the PD-L1 axis has been determined to be increased in the cell or in the subject.

When the immune checkpoint modulating agent is an antibody, it can be designed to be specific to one polypeptide (e.g., monospecific) or having a plural specificity to more than one polypeptide as described herewith. The immune checkpoint modulating agent can be a single type of antibody for a single immune response stimulating target or a combination of more than one antibody each specific for the same or a different immune response stimulating target(s). For example, the immune checkpoint modulating agent can include one, two, three, four, five, or more different antibodies which can be specific to one, two, three, four, five, or more different immune response stimulating targets.

In additional embodiments, the immune checkpoint modulating agent can be a viral infection (e.g., oncolytic viral infection, for example, by talimogene laherparepvec; T-VEC)), an adoptive cellular therapy (e.g., an adoptive cell therapy with chimeric antigen receptor (CAR)-expressing T cells, an adoptive cell therapy with transgenic T cell receptor (TCR)-expressing T cells, an adoptive cell therapy with autologous tumor-infiltrating T cells and/or an adoptive cell therapy with allogeneic natural killer cells), a small molecule (e.g., adenosine receptor A2A antagonist, indoleamine 2, 3-dioxygenase (IDO) inhibitors, tryptophan-2,3-dioxygenase (TDO) inhibitors, arginase 1 inhibitors), a tumor vaccine (e.g., comprising tumor cells, antigen-presenting cells and/or mutated tumor antigenic peptides), an agonist to Toll-like receptors, an agonist to STING (stimulator of interferon genes), an anti-transforming growth factor-β antibody and/or a bi-specific antibody that redirect natural killer cell or T cell cytotoxicity to a defined tumor antigen or a combination of defined tumor antigens.

### EXAMPLES

### Example 1

The quantity in the PD-L1 axis in immune-cold colorectal cancer (CRC) tumors using multiplexed immuno-multiple reaction monitoring (iMRM) was first determined. The PD-L1 axis proteins were quantified in formalin-fixed paraffin-embedded (FFPE) cores that were punched from tumor tissues of patient-derived xenografts (PDX) models of metastatic CRC. Although CRC tumors are “immune-cold” with only low levels of immune-cell infiltration, which leads to very low expression levels in the PD-L1 axis and makes immune-checkpoint inhibitors ineffective, most in the PD-L1 axis components could be quantified in the different tumors, and low to moderate heterogeneity of protein concentrations could be observed across the three cores that were analyzed per tumor.

Recombinant proteins were proteolytically digested with trypsin and analyzed by LC-MS. Per protein, 3-5 proteotypic peptides were selected based on several criteria. This include peptides that were not excessively hydrophobic, contain no easily modifiable amino acids or amino acids known to be modified in the target proteins (with the exception of the known glycosylation sites being covered in the assay), have a high MS signal response, desirable chromatographic behavior, and that are easy to synthesize and purify. For the selected peptides sequences, synthetic standards were generated (light, SIS).

LC-MRM (and MALDI) parameters were optimized for each peptide: e.g., such parameters include chromatographic separation, choice of MRM transitions and collision energies. The lower limit of detection, the lower limit of quantitation (LLoQ), and the linear range were determined for each of the 26 synthesized peptide pairs.

LC-MRM assays were validated in two non-small cell lung cancer (NSCLC) samples without prior immuno-enrichment. Some peptides could be directly quantified, such as LIATTAHER (SEQ ID NO: 25), LQDAGVYR (SEQ ID NO: 15), NIIQFVHGEEDLK (SEQ ID NO: 16) and ITFPGLHELVR (SEQ ID NO: 2).

Some peptides were not subsequently used for various reasons. This includes those that were not sensitive enough (LLOQ >6 fmol by LC-MRM and/or LLOQ >10 fmol by MALDI), those that have a chromatographic peak shape that was abnormal, and/or in which the chromatography was unstable, and/or undetectable by MALDI. Examples of such peptides include:
(i) EDPSAVPVFSVDYGELDFQWR (SEQ ID NO: 6): LLoQ >60 fmol by LC-MRM and LLoQ >10 fmol by MALDI, peptide was unstable in solution (signal decreased over multiple injections from the same sample), broad peak by LC-MRM, unstable elution.
(ii) ESESTAGSFSLSVR (SEQ ID NO: 1): large peaks, unstable chromatography, other peptides for this protein were more reliable with comparable/better analytical parameters.
(iii) HDSGDQDINVVSTYISK (SEQ ID NO: 5): LLoQ impossible to determine (CV >25% at all concentrations tested), undetectable by MALDI.

Although many cell lines were analyzed for PD-L1 alone, no single cell line expresses all targets, as PD-1, LCK and ZAP70 are native to immune cells while PD-L1, PD-L2 and NT5E should be mainly found in tissues/cancer cells.

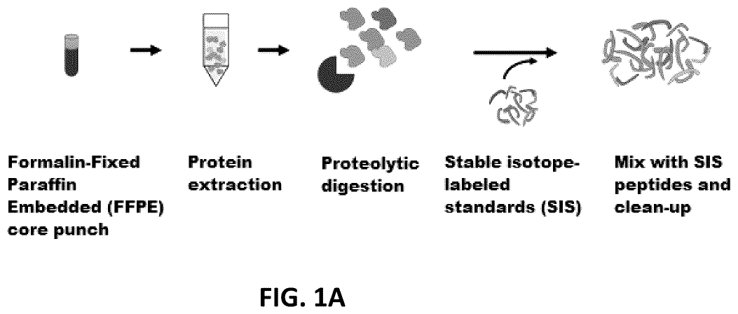

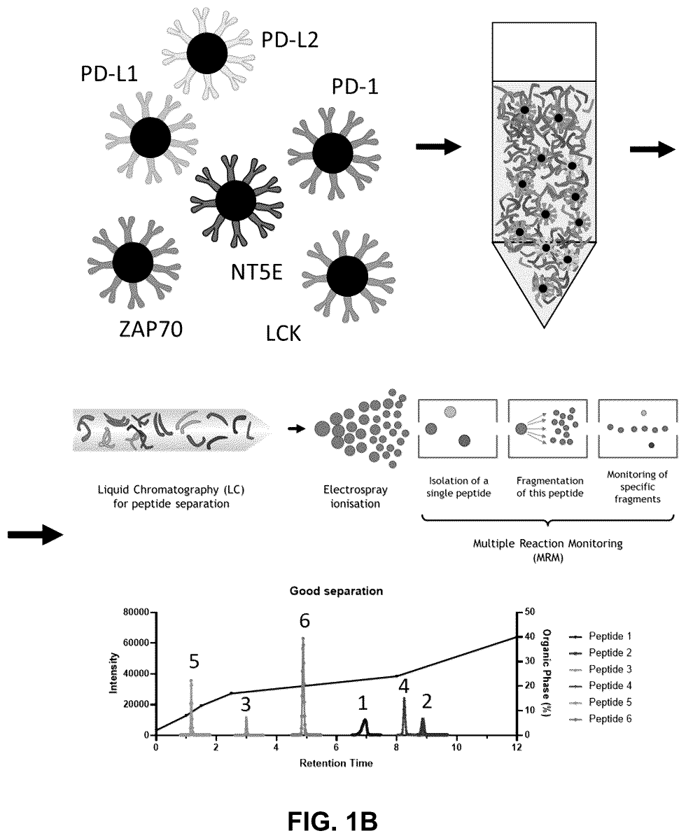

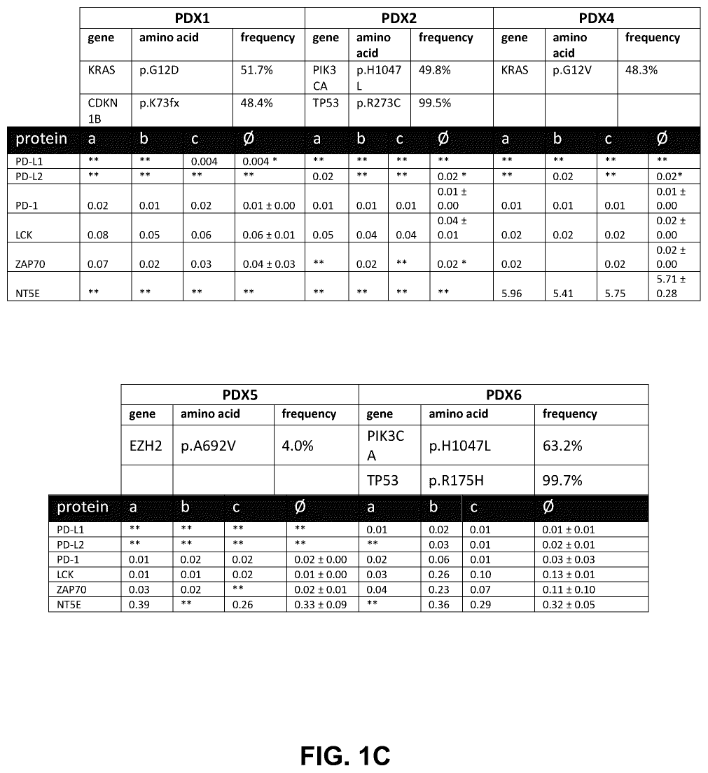

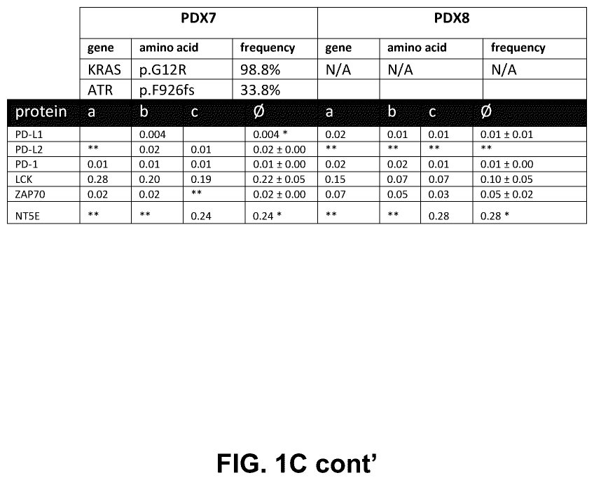

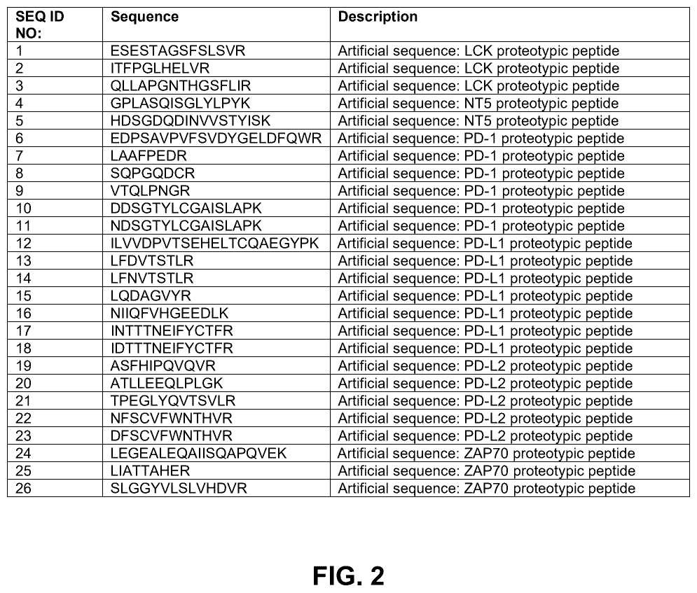

Formalin-fixed paraffin embedded (FFPE) cores of metastatic colorectal cancer (mCRC) tissue were deparaffinized, and protein extracted, followed by proteolytic digestion with trypsin based on filter-aided sample preparation (FASP). 80 fmol of each SIS (1 SIS per target protein) were added to 70 μg of total digested protein (concentration determined using a bicinchoninic acid (BCA) assay). Each sample was vortexed and dried under vacuum. The peptides were resuspended in 150 μL of PBS-C buffer (phosphate-buffered saline buffer+0.015% (w:w) CHAPS). Antibody-bead conjugates were prepared individually by incubating each antibody with protein G magnetic beads for 1 h at room temperature (0.4 μg of antibody per 1 μL of beads). The six antibody-bead conjugates were pooled in a 1:1:1:1:1:1 ratio, the supernatant was removed and the antibody-bead conjugates were resuspended in PBS-C to reach a final concentration of 0.1 μg/μL for each antibody. An equivalent of 1 μg per antibody (6 μg total) was added to the SIS-spiked sample digests. Per sample, the volume was adjusted to 200 μL with PBS-C and samples were incubated overnight at 4° C. while shaking (1300 rpm). The next day, the supernatants were removed and the beads were washed sequentially with 200 μL of PBS-C, 0.1×PBS-C and ultra-pure water. The beads were then transferred to a new tube and the peptides were eluted using 25 μL of 5% acetic acid, 3% acetonitrile at RT for 5 min, while shaking at 1300 rpm. Eluted peptides were vacuum-dried and resuspended in 16 μL of 0.1% formic acid. 15 μL of eluted peptides were injected on an Agilent 1290 infinity LC (2.1×150 mm, 1.8 μm particle size, Zorbax C18) coupled online to an Agilent 6495 triple quadrupole mass spectrometer. An 18-min gradient ranging from 1% to 40% acetonitrile in 0.1% formic acid was used for chromatographic separation. Scheduled multiple reaction monitoring (MRM) was used for determining peak intensities of the endogenous target peptides and their SIS spike-ins. The top 3 most sensitive transitions were used for each peptide and their respective SIS. For data analysis, the Skyline software was combined with custom-made R scripts to quantify endogenous target protein levels based on an external calibration curves that were acquired with the same (constant) amounts of SIS peptides loaded on-column. The results are shown on FIG. 1.

### Example 2

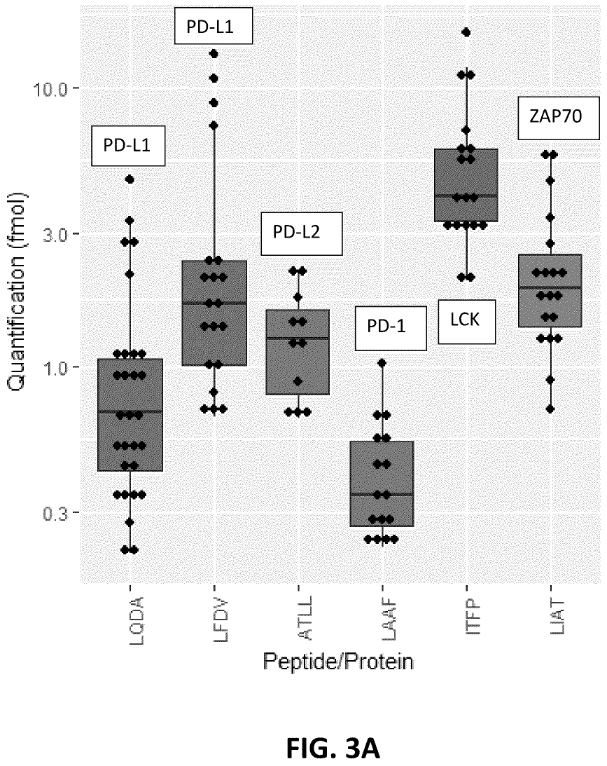

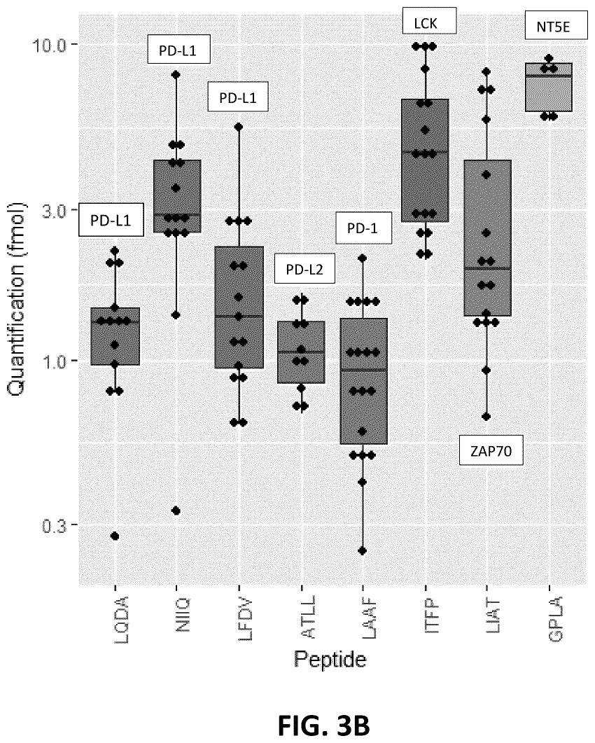

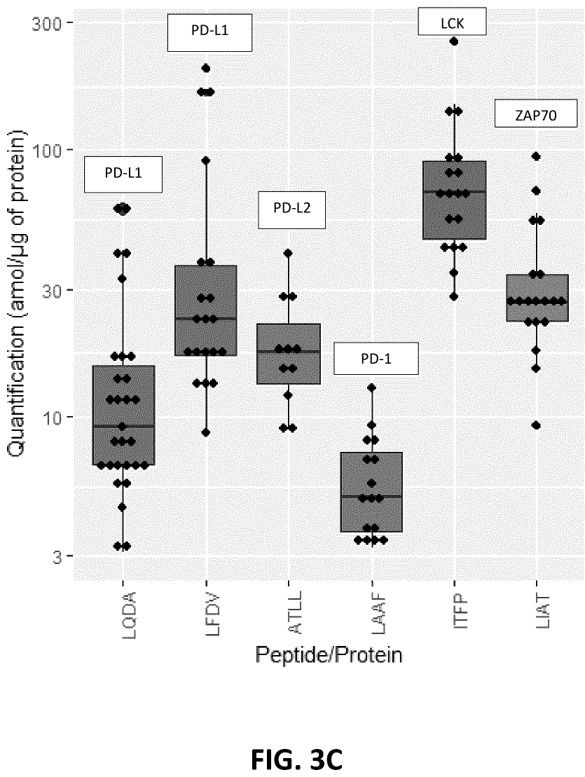

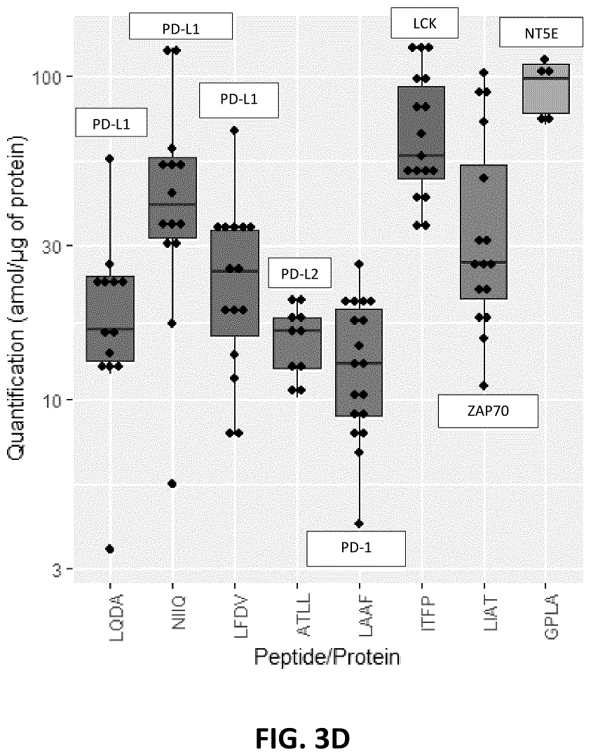

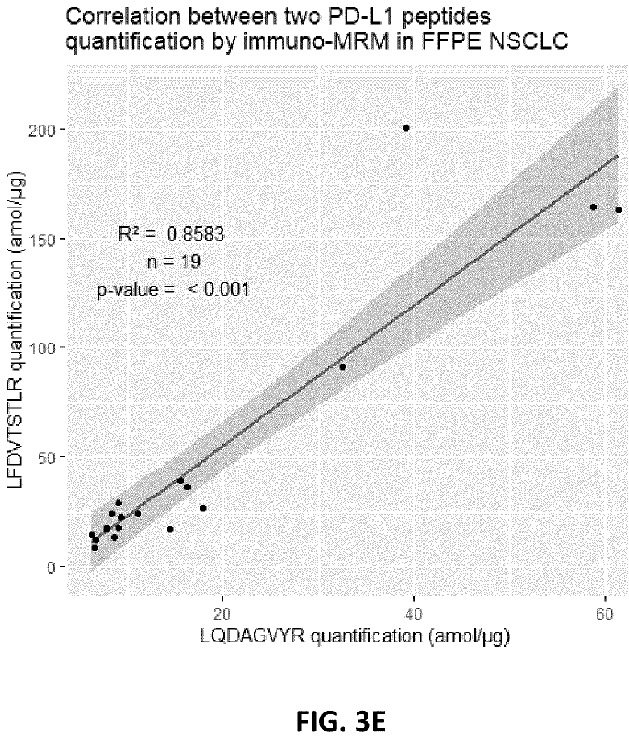

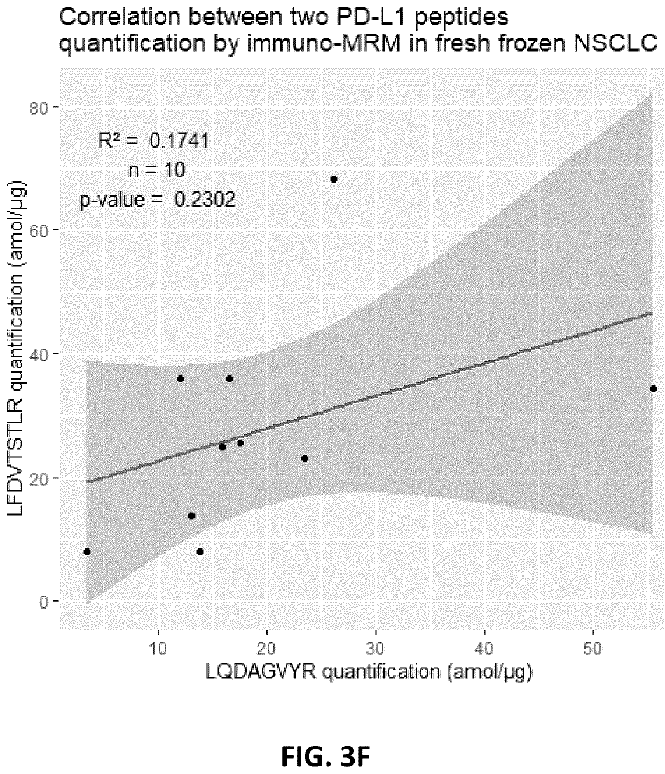

Samples from 38 non-small cell lung cancer (NSCLC) tumors obtained from patients were measured using the assay of Example 1. This included 26 formalin-fixed paraffin embedded (FFPE) samples and 22 samples frozen immediately following extraction (fresh frozen), 10 of which were matched FFPE and fresh frozen. The results are shown in FIG. 3.

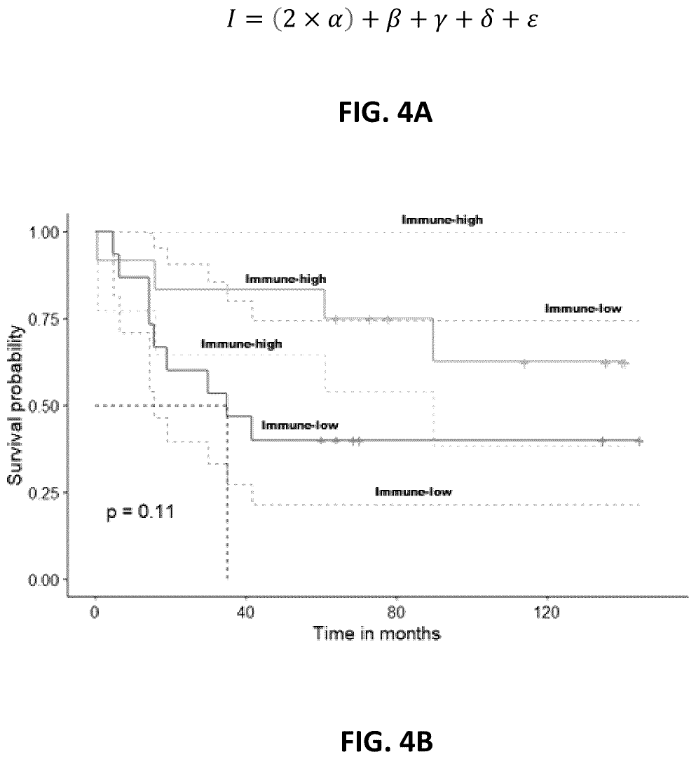

The inventors designed a mathematical model from the quantification data that they obtained. The mathematical model was based on the correlation between survival and the concentration of each peptide (FIG. 4A). The result is an “immunoscore”, which can either be read as a continuous value (from −6 to +6) or a discrete value (immune-high I≥0, immune-low I<0).

The data suggests that the immune-high cluster potentially has a better survival (see Kaplan Meier curve of FIG. 4B). The “immune-low” group (as indicated) had an average survival of 48 months while the “immune-high” (in blue) group had an average survival of 87 months. (p-value=0.11).

The PD-L2, LCK and ZAP70 were the proteins most correlated with survival, suggesting they contribute significantly to the potential prognostic value of the assay.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Ibrahim S, Froehlich B C, Aguilar-Mahecha A, Aloyz R, Poetz O, Basik M, Batist G, Zahedi R P, Borchers CH. Using Two Peptide Isotopologues as Internal Standards for the Streamlined Quantification of Low-Abundance Proteins by Immuno-MRM and Immuno-MALDI. Anal Chem. 2020 Sep. 15; 92(18):12407-12414. doi: 10.1021/acs.analchem.0c02157. Epub 2020 September 1. PMID: 32786432.

1. A combination of proteotypic peptides for quantitative mass spectrometry for at least one protein in a programmed death-ligand 1 (PD-L1) axis, wherein
the at least one protein in the PD-L1 axis is lymphocyte specific kinase (LCK), NT5E (CD73), programmed cell death protein 1 (PD-1), programmed death-ligand 1 (PD-L1), programmed death-ligand 2 (PD-L2) or zeta-chain-associated protein kinase 70 (ZAP70);
the combination comprises at least two of the following synthetic standard peptides:
(i) a synthetic standard lymphocyte specific kinase (LCK) peptide:

| (SEQ ID NO: 1) | |
|---|---|
| | ESESTAGSFSLSVR, |
| (SEQ ID NO: 2) | |
| | ITFPGLHELVR |
| | and/or |
| (SEQ ID NO: 3) | |
| | QLLAPGNTHGSFLIR; |

(ii) a synthetic standard NT5E (CD73) peptide: GPLASQISGLYLPYK (SEQ ID NO: 4) and/or HDSGDQDINVVSTYISK (SEQ ID NO: 5);
(iii) a synthetic standard programmed cell death protein 1 (PD-1) peptide: EDPSAVPVFSVDYGELDFQWR (SEQ ID NO: 6); LAAFPEDR (SEQ ID NO: 7); SQPGQDCR (SEQ ID NO: 8); VTQLPNGR (SEQ ID NO: 9); DDSGTYLCGAISLAPK (SEQ ID NO: 10) and/or NDSGTYLCGAISLAPK (SEQ ID NO: 11);
(iv) a synthetic standard programmed death-ligand 1 (PD-L1) peptide:

| (SEQ ID NO: 12) | |
|---|---|
| | ILVVDPVTSEHELTCQAEGYPK; |
| (SEQ ID NO: 13) | |
| | LFDVTSTLR; |
| (SEQ ID NO: 14) | |
| | LFNVTSTLR; |
| (SEQ ID NO: 15) | |
| | LQDAGVYR; |
| (SEQ ID NO: 16) | |
| | NIIQFVHGEEDLK; |
| (SEQ ID NO: 17) | |
| | INTTTNEIFYCTFR |
| | and/or |
| (SEQ ID NO: 18) | |
| | IDTTTNEIFYCTFR; |

(v) a synthetic standard programmed death-ligand 2 (PD-L2) peptide:

| (SEQ ID NO: 19) | |
|---|---|
| | ASFHIPQVQVR; |
| (SEQ ID NO: 20) | |
| | ATLLEEQLPLGK, |
| (SEQ ID NO: 21) | |
| | TPEGLYQVTSVLR; |
| (SEQ ID NO: 22) | |
| | NFSCVFWNTHVR |
| | and/or |
| (SEQ ID NO: 23) | |
| | DFSCVFWNTHVR; |
| | or |

(vi) a synthetic standard zeta-chain-associated protein kinase 70 (ZAP70) peptide: LEGEALEQAIISQAPQVEK (SEQ ID NO: 24); LIATTAHER (SEQ ID NO: 25) and/or SLGGYVLSLVHDVR (SEQ ID NO: 26).

2. The combination of claim 1, comprising synthetic standard peptides specific for at least two proteins in the PD-L1 axis.

3. The combination of claim 1, wherein each of the synthetic standard peptides comprise an epitope that is present on the corresponding protein in the PD-L1 axis.

4. The combination of claim 1 comprising the synthetic standard PD-L1 peptide.

5. The combination of claim 1 comprising unlabeled synthetic standard peptides, isotope-labeled synthetic standard peptides, or a combination of both.

6. The combination of claim 1 further comprising at least one endogenous peptide obtained from the proteolytic digestion of LCK, NT5E, PD-1, PD-L1, PD-L2 or ZAP70.

7. The combination of claim 1 comprising a deglycosylated proteotypic standard synthetic peptide and/or a deglycosylated endogenous peptide.

8. The combination of claim 1, wherein the quantitative mass spectrometry is a multiple reaction monitoring mass spectrometry, a parallel reaction monitoring mass spectrometry, a matrix-assisted laser desorption/ionization (MALDI) mass spectrometry, or a data independent acquisition mass spectrometry.

9. The combination of claim 8, wherein the multiple reaction monitoring mass spectrometry is an immuno-multiple reaction monitoring mass spectrometry, an immune-parallel reaction monitoring, an immune-MALDI, or an immuno-data independent acquisition mass spectrometry.

10. An antibody combination for use as affinity agents prior to a quantitative mass spectrometry, wherein the antibody combination comprises at least two antibodies and each antibody is able to bind to a different synthetic standard peptide defined in claim 1.

11. The antibody combination of claim 10, wherein at least one antibody of the combination has been obtained by eliciting antibody production in a subject having been administered an immunogen comprising the amino acid sequence of a combination of synthetic standard peptides selected from:
a synthetic standard lymphocyte specific kinase (LCK) peptide: ESESTAGSFSLSVR (SEQ ID NO: 1), ITFPGLHELVR (SEQ ID NO: 2) and QLLAPGNTHGSFLIR (SEQ ID NO: 3
a synthetic standard NT5E (CD73) peptide: GPLASQISGLYLPYK (SEQ ID NO: 4) and HDSGDQDINVVSTYISK (SEQ ID NO: 5);
a synthetic standard programmed cell death protein 1 (PD-1) peptide:

| (SEQ ID NO: 6) | |
|---|---|
| | EDPSAVPVFSVDYGELDFQWR; |
| (SEQ ID NO: 7) | |
| | LAAFPEDR; |
| (SEQ ID NO: 8) | |
| | SQPGQDCR; |
| (SEQ ID NO: 9) | |
| | VTQLPNGR; |
| (SEQ ID NO: 10) | |
| | DDSGTYLCGAISLAPK |
| | and |
| (SEQ ID NO: 11) | |
| | NDSGTYLCGAISLAPK; |

a synthetic standard programmed death-ligand 1 (PD-L1) peptide:

| (SEQ ID NO: 12) | |
|---|---|
| | ILVVDPVTSEHELTCQAEGYPK; |
| (SEQ ID NO: 13) | |
| | LFDVTSTLR; |
| (SEQ ID NO: 14) | |
| | LFNVTSTLR; |
| (SEQ ID NO: 15) | |
| | LQDAGVYR; |
| (SEQ ID NO: 16) | |
| | NIIQFVHGEEDLK; |
| (SEQ ID NO: 17) | |
| | INTTTNEIFYCTFR |
| | and |
| (SEQ ID NO: 18) | |
| | IDTTTNEIFYCTFR; |

a synthetic standard programmed death-ligand 2 (PD-L2) peptide: ASFHIPQVQVR (SEQ ID NO: 19): ATLLEEQLPLGK (SEQ ID NO: 20), TPEGLYQVTSVLR (SEQ ID NO: 21): NFSCVFWNTHVR (SEQ ID NO: 22) and DFSCVFWNTHVR (SEQ ID NO: 23); and/or
a synthetic standard zeta-chain-associated protein kinase 70 (ZAP70) peptide:

| (SEQ ID NO: 24) | |
|---|---|
| | LEGEALEQAIISQAPQVEK; |
| (SEQ ID NO: 25) | |
| | LIATTAHER |
| | and |
| (SEQ ID NO: 26) | |
| | SLGGYVLSLVHDVR. |

12. The antibody combination of claim 10 comprising at least one monoclonal antibody, a polyclonal antibody or a single domain antibody.

13. The antibody combination of claim 10, wherein the quantitative mass spectrometry is a multiple reaction monitoring mass spectrometry, a parallel reaction monitoring mass spectrometry, a matrix-assisted laser desorption/ionization (MALDI) mass spectrometry, or a data-independent acquisition mass spectrometry.

14. The antibody combination of claim 13, wherein the multiple reaction monitoring mass spectrometry is an immuno-multiple reaction monitoring mass spectrometry, an immune-parallel reaction monitoring, an immune-MALDI, or an immuno-data independent acquisition mass spectrometry.

15. A kit comprising the antibody combination of claim 10.

16. A method of performing a quantitative mass spectrometry to determine the abundance of at least two proteins in a programmed death-ligand 1 (PD-L1) axis in a biological sample from a subject, the method comprises:
a) obtaining a biological sample suspected of comprising endogenous peptides obtained from the proteolytic digestion of the at least two proteins in the PD-L1 axis;
b) combining the sample with a combination of at least two synthetic standard peptides, each synthetic standard peptide being specific for a different protein in the PD-L1 axis to obtain a supplemented mixture;
c) enriching the supplemented mixture with a combination of at least two affinity agents, wherein the combination comprises at least one affinity agent being specific for each synthetic standard peptide to obtain an enriched mixture; and
d) submitting the enriched mixture to the quantitative mass spectrometry to determine the abundance of the at least two proteins in the PD-L1 axis;
wherein the proteins in the PD-L1 axis comprises LCK, NT5E, PD-1, PD-L1, PD-L2 and ZAP70.

17. The method of claim 16, wherein the at least two synthetic standard peptides are defined in any one of claims 1 to 7.

18. The method of claim 16, wherein the combination of the at least two affinity agents comprises at least two antibodies.

19. The method of claim 17, wherein the combination of the at least two antibodies is defined in any one of claims 10 to 12.

20. The method of claim 16, further comprising, prior to step a), obtaining the biological sample from the subject.

21. The method of claim 16, further comprising, prior to step a), enzymatically treating the biological sample form the subject to obtain the endogenous peptides.

22. The method of claim 16, further comprising, before step c), deglycosylating the synthetic standard peptides and/or the endogenous peptides.

23. The method of claim 16, wherein the biological sample comprises a fluid, a cell or a tissue.

24. The method of claim 23, wherein the biological sample comprises a living cell, a frozen cell or a fixed cell.

25. The method of claim 16, wherein the biological sample is suspected of comprising a cancerous cell.

26. The method of claim 16, wherein the quantitative mass spectrometry is a multiple reaction monitoring mass spectrometry, a parallel reaction monitoring mass spectrometry, a matrix-assisted laser desorption/ionization (MALDI) mass spectrometry or a data-independent acquisition mass spectrometry.

27. The method of claim 26, wherein the multiple reaction monitoring mass spectrometry is an immuno-multiple reaction monitoring mass spectrometry, an immune-parallel reaction monitoring, an immune-MALDI, or an immuno-data independent acquisition mass spectrometry.

28. (canceled)

29. A method of treating a subject having or suspected to have a condition associated with a modulation in the expression of a protein in a programmed death-ligand 1 (PD-L1) axis, the method comprising determining the abundance of at least two proteins in a programmed death-ligand 1 (PD-L1) axis in a biological sample of the subject and administering an immune checkpoint inhibitor or an immune checkpoint inhibitor activating agent to the subject when it has been determined that the biological sample comprises a modulation in the expression of at least two proteins in the PD-L1 axis, wherein the proteins in the PD-L1 axis comprises LCK, NT5E, PD-1, PD-L1, PD-L2 and ZAP70.

30. The method of claim 29, wherein the condition is a cancer.

31. The method of claim 30, wherein the condition is a solid cancer.

32. The method of claim 31, wherein the solid cancer is lung cancer.

33. The method of claim 29 comprising of
a) obtaining the biological sample suspected of comprising endogenous peptides obtained from the proteolytic digestion of the at least two proteins in the PD-L1 axis;
b) combining the sample with a combination of at least two synthetic standard peptides, each synthetic standard peptide being specific for a different protein in the PD-L1 axis to obtain a supplemented mixture;
c) enriching the supplemented mixture with a combination of at least two affinity agents, wherein the combination comprises at least one affinity agent being specific for each synthetic standard peptide to obtain an enriched mixture; and
d) submitting the enriched mixture to the quantitative mass spectrometry to determine the abundance of the at least two proteins in the PD-L1 axis.

34. The method of claim 33, wherein the subject did not previously receive administering the immune checkpoint inhibitor or the immune check-point inhibitor activating agent.

35. The method of claim 34, wherein the subject did previously receive administering the immune checkpoint inhibitor or the immune check-point inhibitor activating agent.

36. The method of claim 35, wherein the immune checkpoint inhibitor activating agent comprises an antagonistic antibody.

37. The method of claim 36, wherein the immune checkpoint inhibitor activating agent comprises an anti-programmed cell death 1 (PD-1) antibody, an anti-programmed cell death 1 ligand (PD-L1) antibody, and/or an anti-programmed cell death 2 ligand (PD-L2) antibody.


SEQUENCE LISTING (26 sequences)

SEQ ID NO: 1 - protein, 14 aa, Homo sapiens
ESESTAGSFSLSVR

SEQ ID NO: 2 - protein, 11 aa, Homo sapiens
ITFPGLHELVR

SEQ ID NO: 3 - protein, 15 aa, Homo sapiens
QLLAPGNTHGSFLIR

SEQ ID NO: 4 - protein, 15 aa, Homo sapiens
GPLASQISGLYLPYK

SEQ ID NO: 5 - protein, 17 aa, Homo sapiens
HDSGDQDINVVSTYISK

SEQ ID NO: 6 - protein, 21 aa, Homo sapiens
EDPSAVPVFSVDYGELDFQWR

SEQ ID NO: 7 - protein, 8 aa, Homo sapiens
LAAFPEDR

SEQ ID NO: 8 - protein, 8 aa, Homo sapiens
SQPGQDCR

SEQ ID NO: 9 - protein, 8 aa, Homo sapiens
VTQLPNGR

SEQ ID NO: 10 - protein, 16 aa, Homo sapiens
DDSGTYLCGAISLAPK

SEQ ID NO: 11 - protein, 16 aa, Homo sapiens
NDSGTYLCGAISLAPK

SEQ ID NO: 12 - protein, 22 aa, Homo sapiens
ILVVDPVTSEHELTCQAEGYPK

SEQ ID NO: 13 - protein, 9 aa, Homo sapiens
LFDVTSTLR

SEQ ID NO: 14 - protein, 9 aa, Homo sapiens
LFNVTSTLR

SEQ ID NO: 15 - protein, 8 aa, Homo sapiens
LQDAGVYR

SEQ ID NO: 16 - protein, 13 aa, Homo sapiens
NIIQFVHGEEDLK

SEQ ID NO: 17 - protein, 14 aa, Homo sapiens
INTTTNEIFYCTFR

SEQ ID NO: 18 - protein, 14 aa, Homo sapiens
IDTTTNEIFYCTFR

SEQ ID NO: 19 - protein, 11 aa, Homo sapiens
ASFHIPQVQVR

SEQ ID NO: 20 - protein, 12 aa, Homo sapiens
ATLLEEQLPLGK

SEQ ID NO: 21 - protein, 13 aa, Homo sapiens
TPEGLYQVTSVLR

SEQ ID NO: 22 - protein, 12 aa, Homo sapiens
NFSCVFWNTHVR

SEQ ID NO: 23 - protein, 12 aa, Homo sapiens
DFSCVFWNTHVR

SEQ ID NO: 24 - protein, 19 aa, Homo sapiens
LEGEALEQAIISQAPQVEK

SEQ ID NO: 25 - protein, 9 aa, Homo sapiens
LIATTAHER

SEQ ID NO: 26 - protein, 14 aa, Homo sapiens
SLGGYVLSLVHDVR